(19) United States
(12) Patent Application Publication
(10) Pub. No.: US 2007/0156032 A1
(43) Pub. Date: Jul. 5, 2007

(54) Electronic disease management system

(76) Inventors: Linda Susan Gordon, Pittsburgh, PA (US); Christopher John Rapier, Pittsburgh, PA (US)

Correspondence Address: PIETRAGALLO, BOSICK & GORDON LLP, ONE OXFORD CENTRE, 38TH FLOOR, 301 GRANT STREET, PITTSBURGH, PA, 15219-6404, US

(21) Appl. No.: 11/649,638

(22) Filed: Jan. 4, 2007

Related U.S. Application Data
(60) Provisional application No. 60/756,032, filed on Jan. 4, 2006.

(51) Int. Cl. A61B 5/00

(52) U.S. Cl. 600/300; 128/920; 128/923

(57) ABSTRACT

An advanced electronic disease management system is disclosed. The system has been designed to provide medical offices and hospitals with easily accessible guidelines for managing primary and secondary prevention of medical conditions, such as vascular disease. The system allows for easy integration with existing electronic data stores as well as newly entered manual and electronic records. The system is suitable for use with well-known computer interface models, and can be rapidly integrated into existing medical practices. The system can include a computer system capable of receiving a plurality of patient information inputs and a plurality of medical standards inputs, and means for comparing the patient information inputs against the medical standards inputs to produce a recommendation output.

### CROSS REFERENCE TO RELATED APPLICATION

This application claims the benefit of U.S. Provisional Patent Application Ser. No. 60/756,032 filed Jan. 4, 2006, which is herein incorporated by reference.

### FIELD OF THE INVENTION

The present invention relates generally to a system and method for processing medical records and medical information, and more particularly, to an electronic disease management system for receiving input data, performing algorithmic interpretations of the data, and producing medically significantly outputs.

### BACKGROUND INFORMATION

Over the next several years, virtually all hospitals and medical practices will be required to have electronic medical records (EMR) capabilities. However, as of today, only 10% to 20% of all hospitals and physician practices have such capabilities. Accordingly, a major push is underway to integrate EMR into the daily routine of medical practices. Additionally, health insurers are directing health care practices to incorporate risk reduction guidelines in an effort to prevent catastrophic events and significant associated costs. It is predicted that those practices that are EMR compliant, will receive higher levels of reimbursement in the form of bonuses or other compensation from health insurers. As such, a product that integrates risk reduction and EMR capabilities would be well positioned to exploit the information technology changes the medical field is experiencing.

There are many medical treatment programs that can provide effective diagnosis and disease management, however, many of these treatments require a comprehensive patient medical record in order to be effective. Often a patient is treated and/or observed at numerous hospitals and/or physicians' offices, making a complete patient profile difficult to compile in a single location. Even when all medical records and patient history is located in a single location, the voluminous nature of the records can also make it difficult to screen patients for particular risk factors. Accordingly, a need remains for a research tool capable of electronically compiling medical data from multiple sources that is capable of synthesizing the data to assist in the diagnosis of patients exhibiting culminating symptoms of certain medical conditions. A need also remains for a research tool capable of synthesizing the compiled medical data to provide a recommended course of treatment based on the specific profile of a given patient.

### SUMMARY OF THE INVENTION

The present invention provides an advanced electronic disease management system. The system has been designed to provide medical offices and hospitals with easily accessible guidelines for managing primary and secondary prevention of medical conditions, such as vascular disease. The system allows for easy integration with existing electronic data stores as well as newly entered manual and electronic records. The system is suitable for use with well-known computer interface models, and can be rapidly integrated into existing medical practices. The system can include a computer system capable of receiving a plurality of patient information inputs and a plurality of medical standards inputs, and means for comparing the patient information inputs against the medical standards inputs to produce a recommendation output. The system can serve as a valuable research tool as a means to encourage best practices compliance, is highly flexible, and is easily adaptable to medical advances between system upgrades and/or installations. The present invention can also be a readily accessible educational tool for patients.

An aspect of the present invention is to provide a computer-assisted disease management system, comprising a computer system capable of receiving a plurality of patient information inputs and a plurality of medical standards inputs and means for comparing the patient information inputs against the medical standards inputs to produce a diagnosis output.

Another aspect of the present invention is to provide a computer-assisted disease management system, comprising a computer system capable of receiving a plurality of patient information inputs and a plurality of medical standards inputs means for generating an interactive questionnaire in response to the previously entered patient information inputs, and means for comparing the patient information inputs against the medical standards inputs to produce a diagnosis output.

Another aspect of the present invention is to provide a computer-implemented method of generating a recommendation output, comprising entering a plurality of patient information inputs into a computer system entering a plurality of medical standards inputs, generating an interactive questionnaire in response to previously entered patient information inputs, comparing the patient information inputs against the medical standards inputs, and generating a recommendation output in response to the comparison of the patient information inputs and the medical standards inputs.

Another aspect of the present invention is to provide a computer-readable medium having stored thereon instructions which, when executed by a processor, cause the processor to perform the steps of comparing patient information inputs against medical standards inputs and generating a recommendation output in response to the comparison of the patient information inputs and the medical standards inputs.

Another aspect of the present invention is to provide a computer-readable medium having stored thereon instructions which, when executed by a processor, cause the processor to perform the steps of generating an interactive questionnaire in response to previously entered patient information inputs comparing the patient information inputs against medical standards inputs and generating a recommendation output in response to the comparison of the patient information inputs and the medical standards inputs.

Another aspect of the present invention is to provide an apparatus, comprising means for receiving a plurality of patient information inputs, means for receiving a plurality of medical standards inputs, means for generating an interactive questionnaire in response to the previously entered patient information inputs, and means for comparing the patient information inputs against the medical standards inputs to produce a diagnosis output.

Another aspect of the present invention is to provide a system, comprising a processor and a memory in communication with the processor, the memory having stored thereon a set of ordered data and instructions which, when executed by the processor, cause the processor to perform the steps of generating an interactive questionnaire in response to previously entered patient information inputs, comparing the patient information inputs against medical standards inputs, and generating a diagnosis output in response to the comparison of the patient information inputs and the medical standards inputs.

Yet another aspect of the present invention is to provide multi-user computer-based disease management system, comprising a data store for storing medical standards inputs and patient information inputs, a computer system capable inputting and extracting medical standards inputs and patient information inputs from the data store, means for allowing user initiated input of medical data into the data store, means for allowing automatic input of medical data into the data store and means for allowing user interrogation and extraction of data from the data store.

These and other aspects of the present invention will be more apparent from the following description.

### BRIEF DESCRIPTION OF THE DRAWINGS

FIG. 1 is a system flow diagram including patient information inputs, medical standards inputs, and multiple recommendation outputs in accordance with an embodiment of the present invention.

FIG. 2 is a physician flow diagram illustrating system access by a physician in accordance with an embodiment of the present invention.

FIG. 3 is a patient flow diagram illustrating system access by a patient in accordance with an embodiment of the present invention.

FIG. 4 is an administrator flow diagram illustrating access by an administrator in accordance with an embodiment of the present invention.

FIG. 5 is a system login page in accordance with an embodiment of the present invention.

FIG. 6 is a patient search screen accessed by a physician in accordance with an embodiment of the present invention.

FIG. 7 is a criteria selection screen accessed by a physician in accordance with an embodiment of the present invention.

FIG. 8 is a system operation screen generated by the system from the criteria selection screen of FIG. 7 in accordance with an embodiment of the present invention.

FIG. 9 is a results display screen generated by the system from the system operation screen of FIG. 8 in accordance with an embodiment of the present invention.

FIG. 10 is an alternative view of a patient search screen accessed by a physician in accordance with an embodiment of the present invention.

FIG. 11 is a patient search screen that has been queried by a physician in accordance with an embodiment of the present invention.

FIG. 12 is a patient result screen generated by the patient search of FIG. 11 in accordance with an embodiment of the present invention.

FIG. 13 is a patient search screen showing a partial query in accordance with an embodiment of the present invention.

FIG. 14 is a patient result screen generated by the patient search screen of FIG. 13 in accordance with an embodiment of the present invention.

FIG. 15 is the top portion of an add patient screen of the system in accordance with an embodiment of the present invention.

FIG. 16 is the bottom portion an add patient screen of the system in accordance with an embodiment of the present invention.

FIG. 17 is a historical assessment screen in accordance with an embodiment of the present invention.

FIG. 18 is a delete assessment screen in accordance with an embodiment of the present invention.

FIG. 19 is a modified historical assessment screen after a deletion of an assessment record in accordance with an embodiment of the present invention.

FIG. 20 is a patient profile screen in accordance with an embodiment of the present invention.

FIG. 21 is a patient drug questionnaire screen in accordance with an embodiment of the present invention.

FIGS. 22a to 22d illustrate a patient medical questionnaire screen in accordance with an embodiment of the present invention.

FIGS. 23a to 23d are physician questionnaire screens in accordance with an embodiment of the present invention.

FIG. 24 is a physician questionnaire screen showing the questionnaire in tabular form in accordance with an embodiment of the present invention.

FIGS. 25a-25e are physician recommendation screens in accordance with an embodiment of the present invention.

FIG. 26 is a dietary input screen in accordance with an embodiment of the present invention.

FIGS. 27a to 27b are detailed dietary screens in accordance with an embodiment of the present invention.

FIG. 28 is an exercise screen in accordance with an embodiment of the present invention.

FIG. 29 is a sample exercise program screen in accordance with an embodiment of the present invention.

FIG. 30 is a Framingham Risk Assessment screen in accordance with an embodiment of the present invention.

FIG. 31 is a patient login screen in accordance with an embodiment of the present invention.

FIG. 32 is a patient access screen in accordance with an embodiment of the present invention.

FIGS. 33a to 33d illustrate patient recommendation screens in accordance with an embodiment of the present invention.

FIG. 34 is an administrator options screen in accordance with an embodiment of the present invention.

FIG. 35 is an administrator page layout screen in accordance with an embodiment of the present invention.

FIG. 36 is a delete user screen in accordance with an embodiment of the present invention.

FIG. 37 is a change user screen in accordance with an embodiment of the present invention.

FIG. 38 is a question management page in accordance with an embodiment of the present invention.

FIG. 39 is a question modifying screen in accordance with an embodiment of the present invention.

FIG. 40 is an add question screen in accordance with an embodiment of the present invention.

FIG. 41 is a diet document administration screen in accordance with an embodiment of the present invention.

FIGS. 42-43 are diet document management pages in accordance with an embodiment of the present invention.

FIG. 44 is an administrator exercise screen in accordance with an embodiment of the present invention.

FIG. 45 is an administrator editing screen for exercise documents in accordance with an embodiment of the present invention.

FIG. 46 is an administrator exercise screen in accordance with an embodiment of the present invention.

FIG. 47 is an administrator pharmaceutical drug class management screen in accordance with an embodiment of the present invention.

FIG. 48 is an administrator drug class editing screen in accordance with an embodiment of the present invention.

FIG. 49 is a create a new drug class screen in accordance with an embodiment of the present invention.

FIGS. 50 and 51 are edit drug interaction rule screens in accordance with an embodiment of the present invention.

FIGS. 52a and 52b are recommendation set management screens in accordance with an embodiment of the present invention.

FIG. 53 is a recommendation modifying screen in accordance with an embodiment of the present invention.

FIGS. 54a and 54b are data integrity screens in accordance with an embodiment of the present invention.

FIG. 55 is a data restore screen in accordance with an embodiment of the present invention.

FIG. 56 is a date-sorted list of data tables screen in accordance with an embodiment of the present invention.

FIG. 57 is a restore screen in accordance with an embodiment of the present invention.

### DETAILED DESCRIPTION

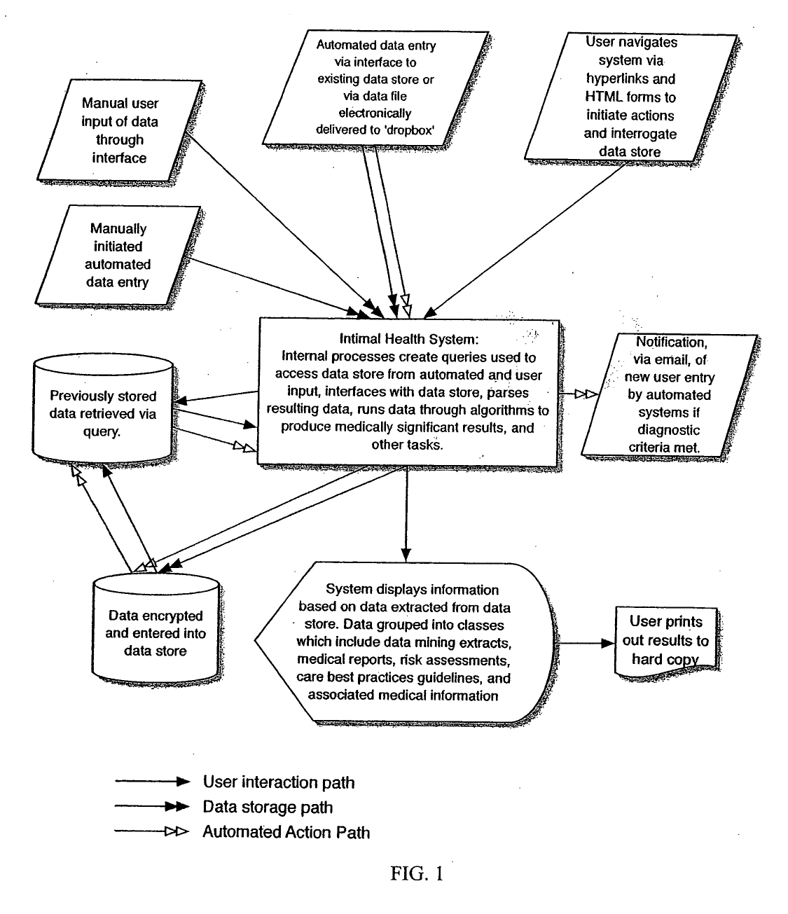

As shown in FIG. 1, the computer-based disease management system of the present invention is capable of receiving a plurality of input information, such as current and/or previous patient information and current medical standards. Previous patient information can include, for example, records from multiple previous treating physicians, previous hospital records, previous prescribed drugs, pharmaceuticals and/or medical devices, and the like. Current patient information can include, for example, information provided real-time by a patient, notes and diagnosis rendered by a current treating physician, and currently prescribed drugs, pharmaceuticals and/or medical devices. Current medical standards can include any literature or treatment regimes accepted by part or all of the medical profession.

As shown in FIG. 1, information can be entered into the system through a data storage path. The input information can be in the form of pre-existing electronic records, newly created electronic records, or manually entered information. Information can be manually entered into the system through a standard computer interface. In another embodiment, automated data entry of information can be manually initiated. In yet another embodiment, data entry can be automated via an interface with an already existing data store. Data entry can also be automated via data files electronically delivered to a computer-driven dropbox. In one embodiment, the disease management system resides at a particular physician's location. Input access to the system can be provided by any conventional network access means, such as, for example, the Internet. In another embodiment, the disease management system can reside partially at a particular physician's location and partially at a centralized server location. In this embodiment, a central server can be in data-communication with a plurality of systems located at a plurality of particular physician locations. It is contemplated herein that standard information security procedures can be implemented with the present invention to maintain privacy and information security.

Also shown in FIG. 1, a system user can navigate the system to initiate system actions and access stored data. The electronic disease management system of the present invention utilizes internal processes to create queries used to access data stores created from automated and manually entered inputs, such as current and/or previous patient information and current medical standards. The electronic disease management system of the present invention also interfaces with the data stores, evaluates for completeness, parses the resulting data, and runs the data through algorithms to produce medically significant results. Completeness of data may be indicated by a color-coding system. The algorithms can be created based on current medical standards, a new treatment regime and/or a physician's synthesis of response from a patient.

Still referring to FIG. 1, multiple users of the electronic disease management system are contemplated herein. Physicians, patients and administrators may each be granted limited or complete access to the system, depending on the desired limitations as will be further discussed herein. In one embodiment, multiple physicians, multiple patients, and/or multiple administrators may access the system. In each case, a user can navigate the system via hyperlinks and/or HTML forms to initiate actions and interrogate data stores. Once the user has instructed the system to return a certain set of information, the system will display the requested information based on specific data extracted from one or more data stores. Data can be grouped into one or more classes which can include data mining extracts, medical reports, risk assessments, best care practice guidelines, and associated medical information. The system can produce one or more recommendation outputs based on the extracted data, including, for example, a recommended course of treatment, prescribed drug or pharmaceutical, a medical device, an operative procedure, a diet and/or exercise program, and the like.

Still referring to FIG. 1, a user can access previously stored data by querying the system. This can include past treatments and whether or not they were successful as well as patient information tending to show whether a particular patient would be a good candidate for a specific treatment. The electronic disease management system can also include data encryption means for encrypting entered data and data stores. It is contemplated herein the electronic disease management system can be HIPPA compliant.

Still referring to FIG. 1, once a user has retrieved the desired information from the system, a hard copy print out can be produced that is specific to the user. In one embodiment, a print out can be tailored to a physician, a patients, and/or an administrator dependent on the restrictions of the system. In another embodiment, a notification can be automatically generated and delivered to a user based on newly entered information if certain diagnostic criteria are satisfied. For example, a user may receive an email warning if a change in lifestyle or medication(s) would place a patient at risk for certain conditions.

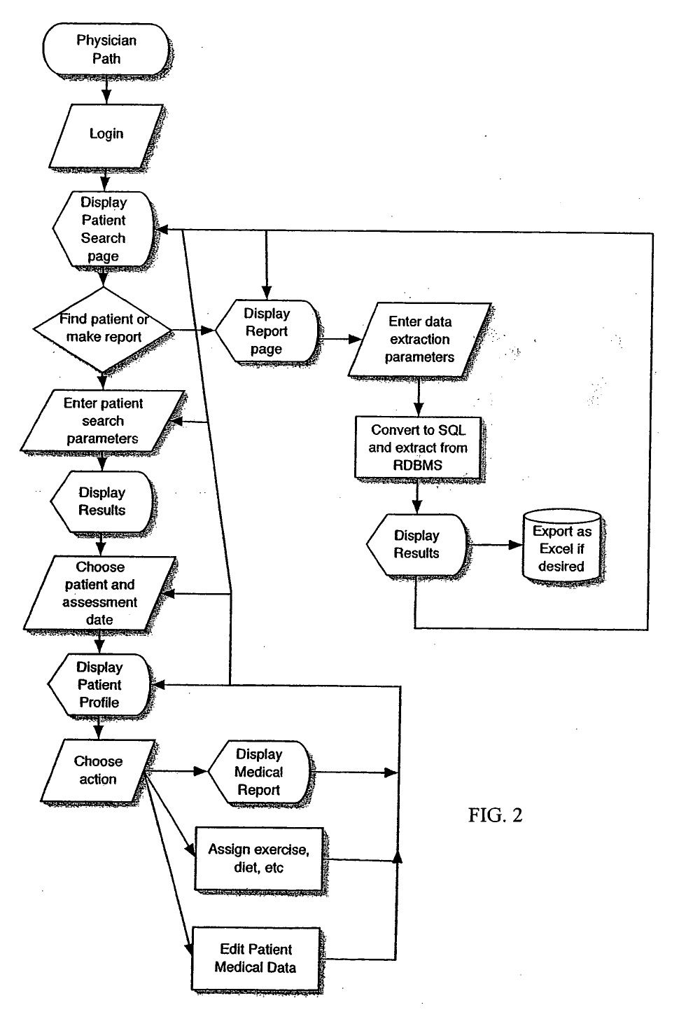

As shown in FIG. 2, if the user is a physician, then physician-relevant aspects of the system will be accessible. For example, the physician may perform a login step from which the physician will have access to a patient database. The physician can find the records of a specific patient, create a new patient record, or create a report. If the physician wishes to access the records of a particular patient, the physician can enter patient search parameters and the system will display data consistent with the entered search terms. From the displayed data the physician can select a particular patient and/or an assessment date(s). Once the system has displayed a specified patient profile, the physician can choose a desired action, such as displaying a medical record for review, assigning a recommended treatment or preventative course of action, and/or edit a patient's medical data. If the physician wishes to make a specific report, the physician can access a report page of the system and enter data extraction parameters in the system. The system can then parse the request against data stores and display the requested recommendation output information. The system routines of the present invention utilize medical information that is specific to an individual patient, and known medical information including previous reactions from that specific individual, or previous reactions from a group of similarly situated patients, in response to a proposed treatment. Example recommendation outputs can include disease diagnosis, prescriptions, drugs and/or pharmaceuticals, medical devices, operative procedures, dietary and/or exercise programs and the like.

Still referring to FIG. 2, in most instances a physician user will have broad access to the data stores to allow a medically comprehensive diagnosis. A physician can have access to multiple patient records. For example, a physician can access multiple patient records for evaluation of the success of a treatment program based on demographics of previous patients. An example physician user action will be shown in the Example included herein.

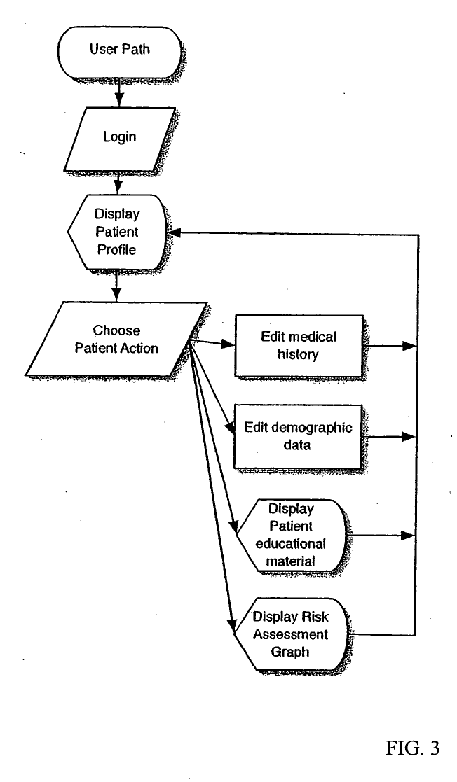

Referring to FIG. 3, if the patient is a user, then patient-relevant aspects of the system will be accessible. In most cases, the information that can be displayed to the patient is less inclusive than the information displayed to a physician. In one embodiment, the information displayed to a patient is restricted to information that is entered by the patient and a course of action that is to be conducted by the patient, such as when to take medications, medication dosage, exercise and dietary programs to be initiated by the patient.

As shown in FIG. 3, a patient may perform a login step from which the patient will have access to that particular patient's profile. From the patient profile, the patient may edit their medical history and/or demographic data to include new information, such as newly discovered family history of disease, recent treatments, and the like. The patient may also request educational material on a condition specific to their profile as well as request a risk assessment graph based on the information specific to their profile. In one embodiment, the system will produce an educational report and/or a risk assessment graph that can be accessed by the patient, however, the system will not allow the patient access to other recommended courses of treatment or previously entered recommended treatments. In another embodiment, a patient will not have access to any other patient's information. In most cases, the information that can be displayed to the patient is less inclusive than the information displayed to a physician. An example patient user action will be shown in the Example included herein.

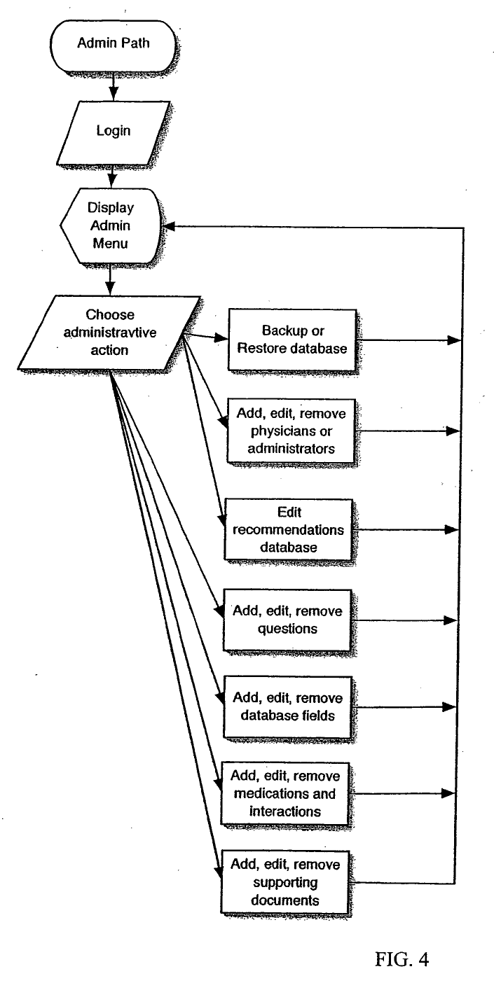

As shown in FIG. 4, if the user is an administrator, then administrator-relevant aspects of the system will be accessible. Once an administrator performs a login step, the administrator can choose to perform a series of administrator actions. An administrator can back-up or restore the system database. An administrator can also add, edit or remove certain administrators or physicians from the approved users system list. In another embodiment, an administrator can also edit the medical standards inputs. As additional medical information becomes available, an administrator can access the system to alter the recommended treatments and/or the factors relevant to prescribing a specific treatment. An administrator can also add, edit or remove questions that are asked to a patient or a physician and/or database fields within the system depending on changing medical standards inputs and/or requested changes by a physician. In another embodiment, an administrator can also add, edit or remove medications and drug interaction warnings depending on changing medical standards inputs. In yet another embodiment, an administrator can add, edit or remove various other supporting documents or fields from the system, however, in certain situations, the administrator may not access individual patient records and/or treatments.

The electronic disease management system of the present invention will be more fully explained with reference to the following example.

### EXAMPLE

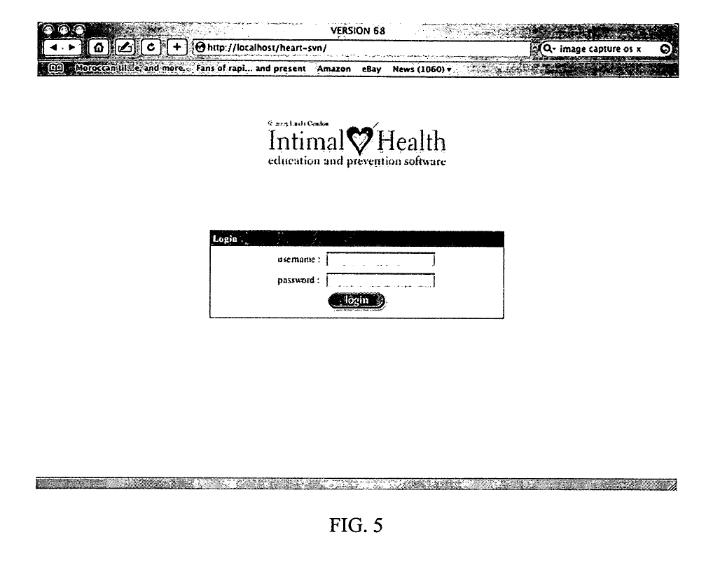

As shown in FIG. 5, the system can include a main login page. An authentication system can be incorporated into the main login page for classifying three distinct categories of users: physicians, patients, and administrators. The entered user name can initially be compared to an authorized user list of physicians and administrators to evaluate whether access is permitted, and if so, what access path is appropriate. If an entered user name matches a user in either the physicians or administrators list, then a subsequently entered password can be compared and verified. In one embodiment, passwords are stored as one-way encrypted SHAI hashes. A clear text password does not need to be transmitted within the system and/or over the network.

If an entered user name does not match a physician or administrator identified on authorized user lists, then the user name is deconstructed to determine if it matches the patient user name format. Patient user names can be assigned by any suitable means, such as composed of the first initial, last name and birth date as a single word. In one example, John Doe born on Dec. 14, 1956 can have a user name ofjdoe12141956. In another embodiment, the password used by Mr. Doe can be the last four digits of his social security number. If the submitted user name and password do not match any of the authorized users, then access is denied. All access attempts can be logged with the attempted user name, time, and results of the attempt. On successful login, a persistent server side file can be created, this is known as a session.

### I Physician Access

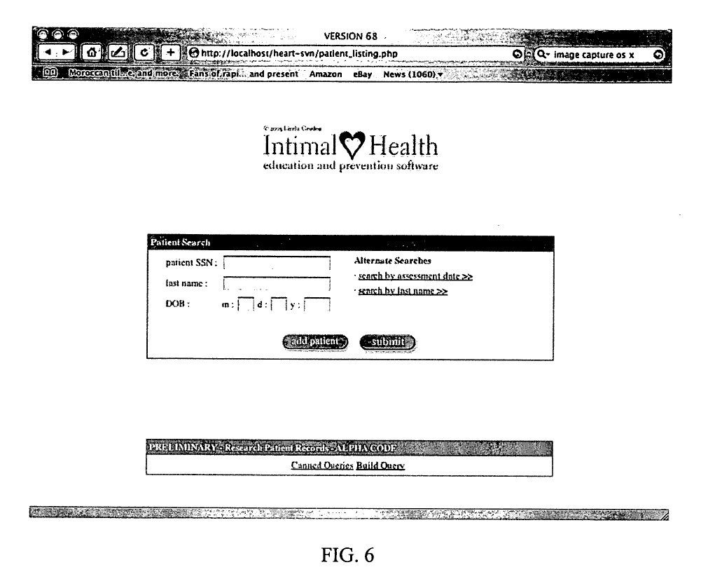

As shown in FIG. 6, when a physician successfully logs in, they can be directed to a Patient Search screen. From this screen, a physician may search for an individual patient or groups of patients based on their name, date of birth, the date a patient evaluation or assessment took place, or by another patient identifier. Combinations of these search characteristics may be combined to refine search results.

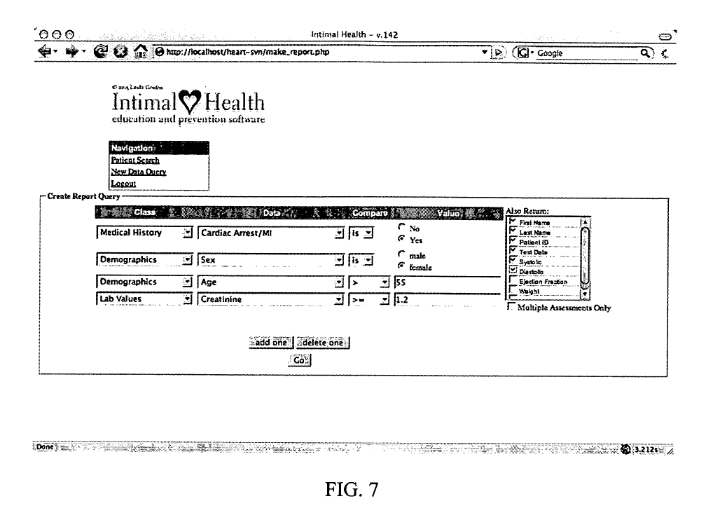

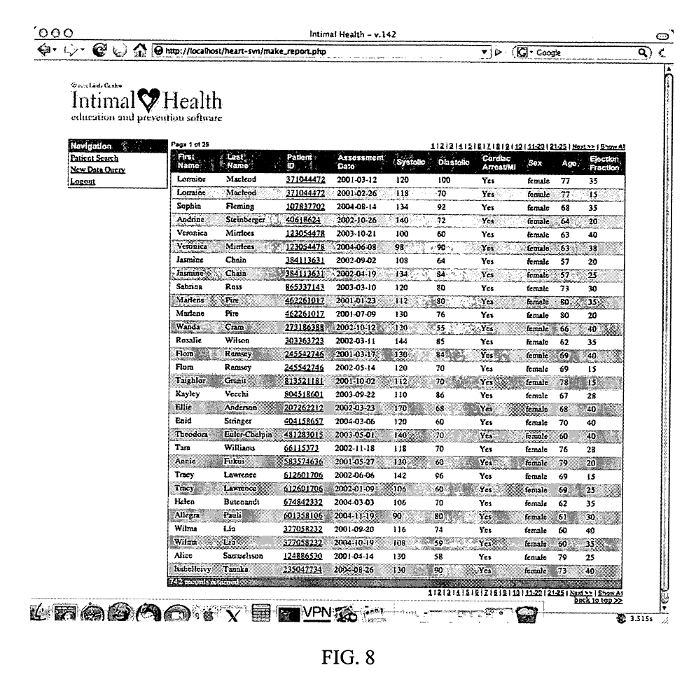

The physician may also create new patient entries such as by using an add patient feature of the system. A physician can run two classes of reports against the medical data stored within the entire patient database. As shown in FIGS. 7 and 8, the physician can evaluate what criteria should be searched through a criteria selection screen and the system can export raw data to a user-friendly results display screen. An example set of search criteria can include a medical history of cardiac arrest in a female having creatinine value of 1.2. In one embodiment, the system can dynamically create, display and/or eliminate object entities of a given document, such as a web document, in response to user actions without reloading or resubmitting the document. If a user selects an element from a drop down menu, then other form elements can be removed and replaced with entirely different object types. Depending on the type of data the user is searching for, the screen will automatically change the presentation of data to display the appropriate form elements. For example, if the desired question is a “yes” or “no” question, then the system can display a yes or no button for the user to select. However, if the same question is changed to one in which a value would be entered by the user, such as “10” or “Bob”, then the system can remove the yes or no button and replace it with a text entry box.

Referring to FIG. 8 and throughout this application, all biographic information and data are fictional values that do not correspond to actual persons.

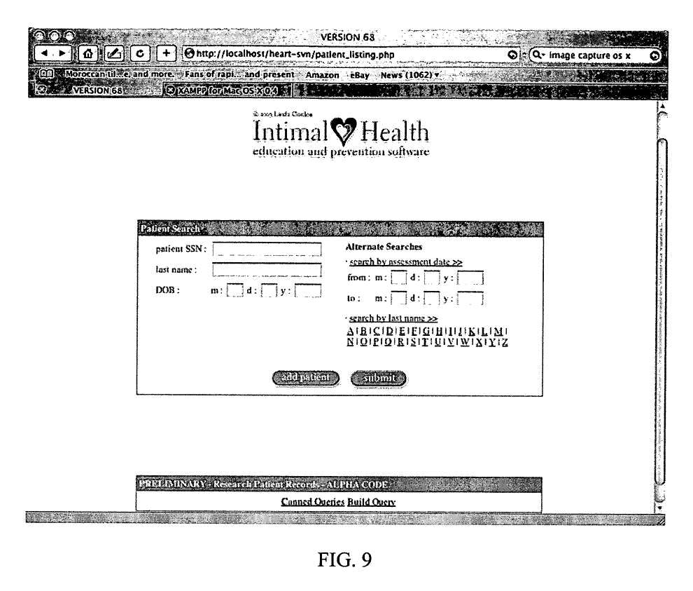

As shown in FIG. 9, an alternative embodiment of the patient search page is shown.

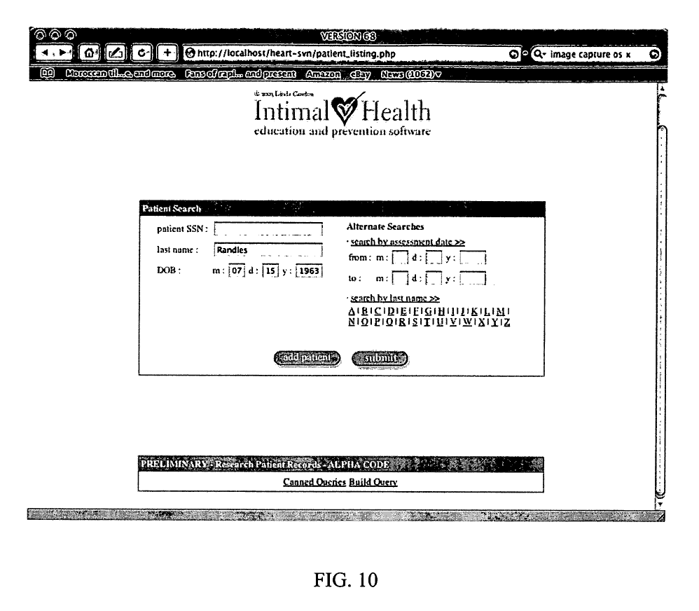

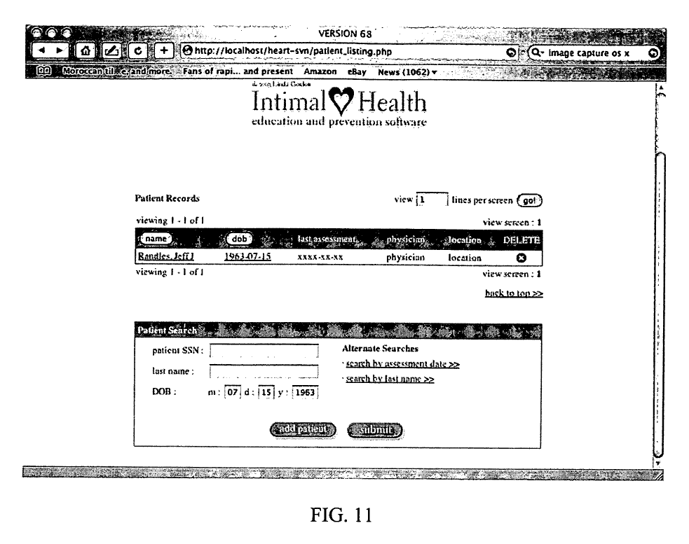

As shown in FIG. 10, a physician can perform a patient search querying the last name and date of birth. This is one of the most common ways for physicians to uniquely identify patients. However, it should be noted that there is no guarantee that this will be sufficient to assure uniqueness in a large hospital system. Accordingly, the present electronic disease management system can also combine one search with another, including queries such as medical record numbers as well as other search criteria. The results of the query shown in FIG. 11 are shown in FIG. 11.

As shown in FIG. 11, a single patient result was returned as a result of a physician querying a patient last name and date of birth. If multiple patients are returned, the physician may reorder them by name, date of birth, the most recent assessment date, primary care physician, the location in which the assessment took place, or other relevant search criteria. It may also be desirable for a physician to delete a patient's medical record from this screen.

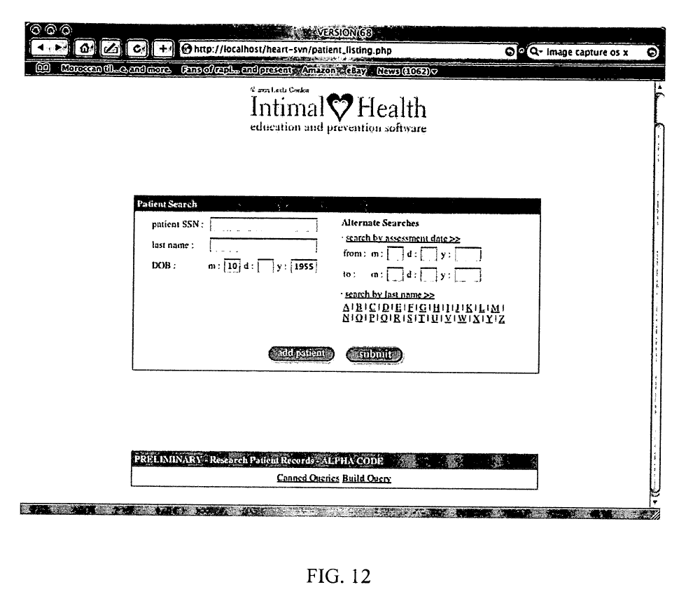

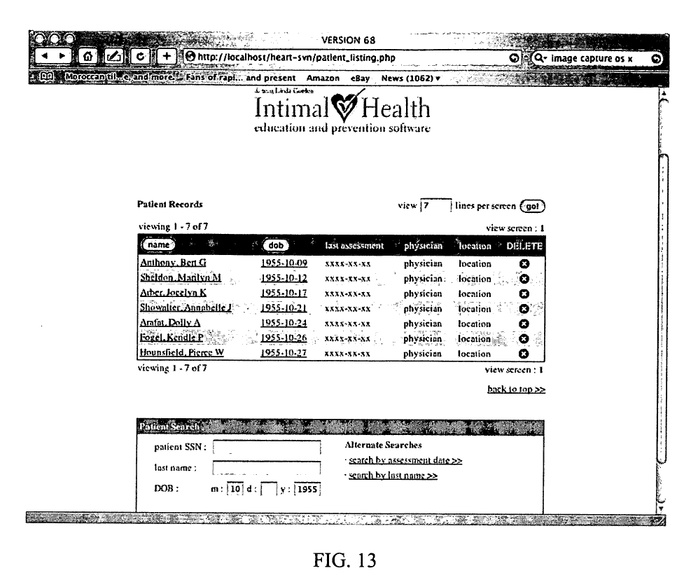

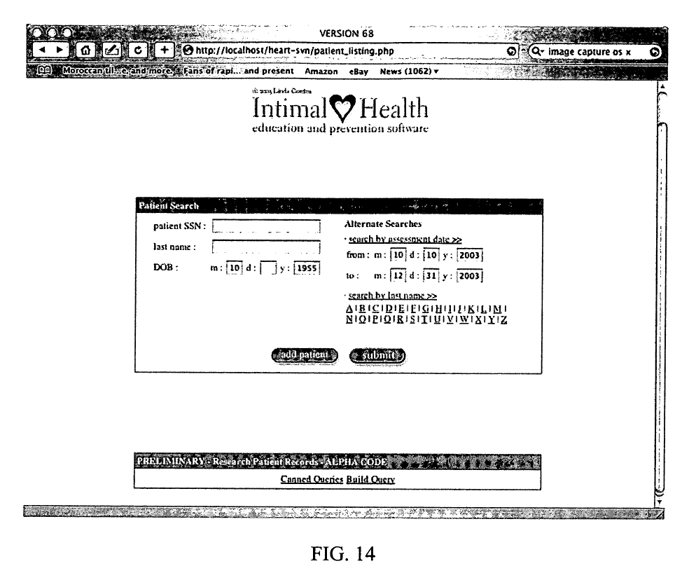

As shown in FIG. 12, the system can also query partial information. In this embodiment, a physician may enter the month and year of birth to retrieve patient statistics. This can return all patients born in a given month of a given year, such as all patients born in October of 1955. Alternatively, a partial search can query the day of the month, the month alone, the year of birth, or any combination of the like. As shown in FIG. 13, the system can return all patients satisfying certain minimum query statistics. This can allow physicians to quickly find patients even if all identifying information isn't available to the physician. Also shown in FIG. 14, is the color-coding system that identifies the level of completeness associated with a selected data set. In this embodiment, the round buttons to the right of the date in the “Last Assessment” column are color-coded as red, yellow or green. Red indicates a critical piece of data is missing from the data set, in this case the last assessment. Yellow indicates the data set is incomplete, but all critical data is available. Green indicates the data set is complete. The color-coding system allows physicians to quickly detect an incomplete record.

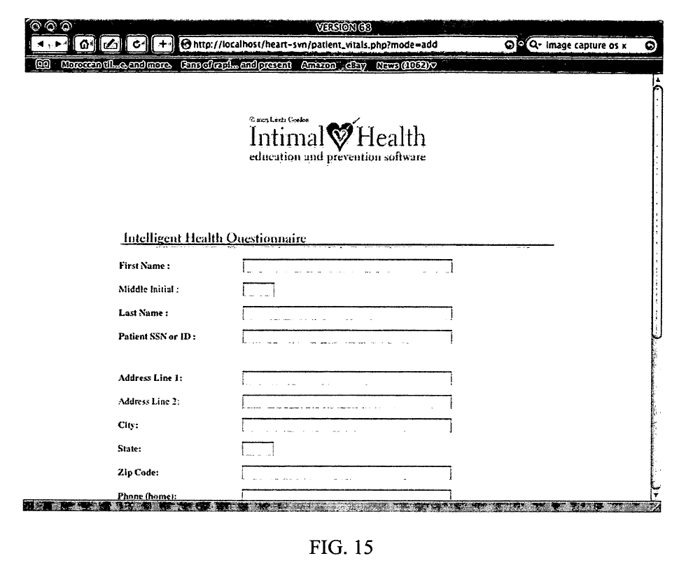

As shown in FIG. 14, a physician's search can be further refined by entering a date range in which a patient assessment took place. As shown in FIG. 15, a search query for all patients born in October of 1955 that were assessed in the last quarter of 2003 is returned.

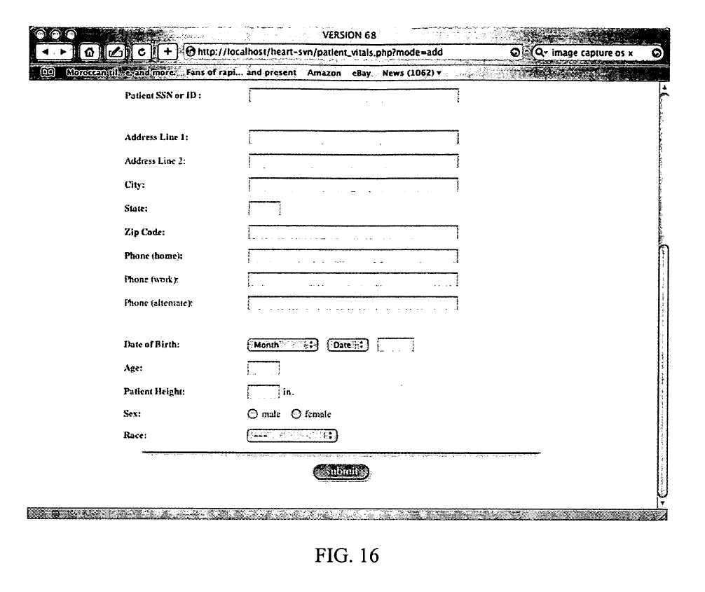

In addition to assessing already existent patient profiles, the present electronic disease management system can be equipped with a feature to allow for the addition of a new patient. As shown in FIGS. 15 and 16, an add patient page allows for the hand entry of new patient demographic information. This information can be used to identify the patient to the physician. This page can also collect information regarding race and gender of a patient. This information can be used later in the program to customize the questions presented to the user as well as recommendations supplied by the system. For example, a question regarding pregnancy would only be presented to female patients. Once patient information has been entered into the page shown in FIGS. 15 and 16, a unique patient ID number can be generated by the system and associated with the patient. This number can be used in all transactions. The patient ID as well as all the associated demographic data is stored in a relational database.

All information that can be used to uniquely identify the patient, such as their name, address, phone numbers, and so forth can be encrypted such as by using a AES 128 bit cipher. In one embodiment, the key used in the encryption process is unique to each installation of the software. A copy of the key can be escrowed by a system administrator to aid in the recovery of data if the user key is lost.

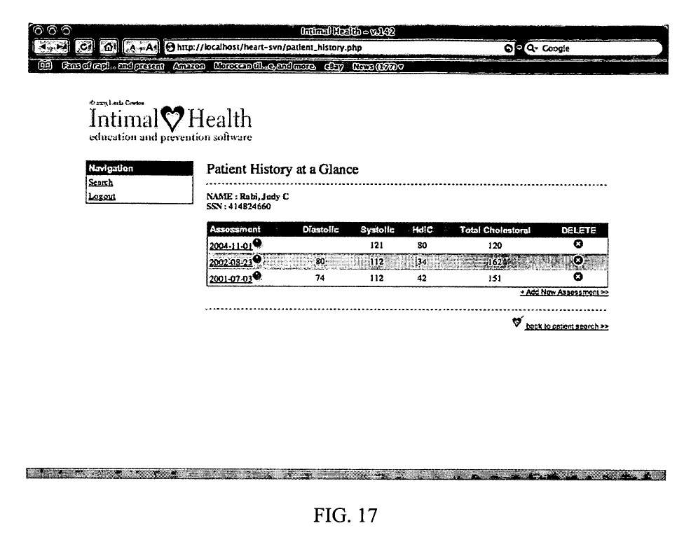

As shown in FIG. 17, once a patient has been selected or created, the internal patient ID number is stored in a server side persistent file known as a session. This will be used throughout the physician's interaction with this patient's records. A screen showing all the historical assessments along with the level of completeness of each assessment can be displayed. If the physician is viewing a new patient, then the physician can be instructed to start the assessment process, and there will be no previous assessments to display. The physician may also add additional assessments to the patient history using an add new assessment link. In one embodiment, a brief synopsis of pertinent information is displayed for each assessment. Example values including blood pressure Hdlc and cholesterol values can be displayed.

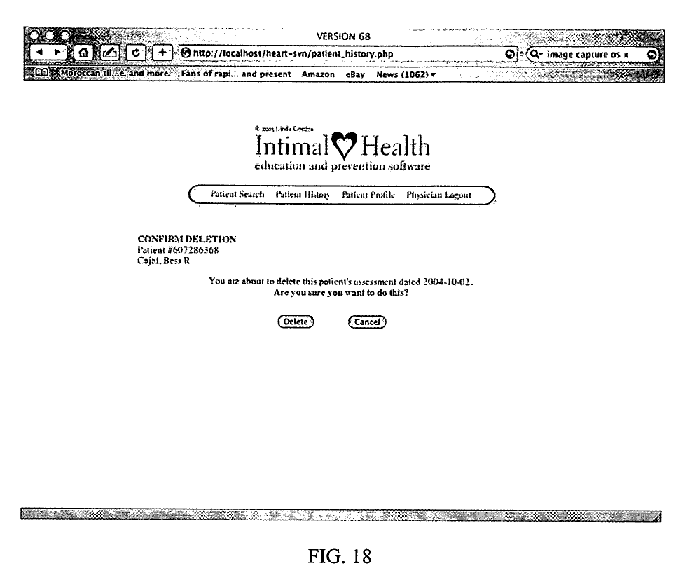

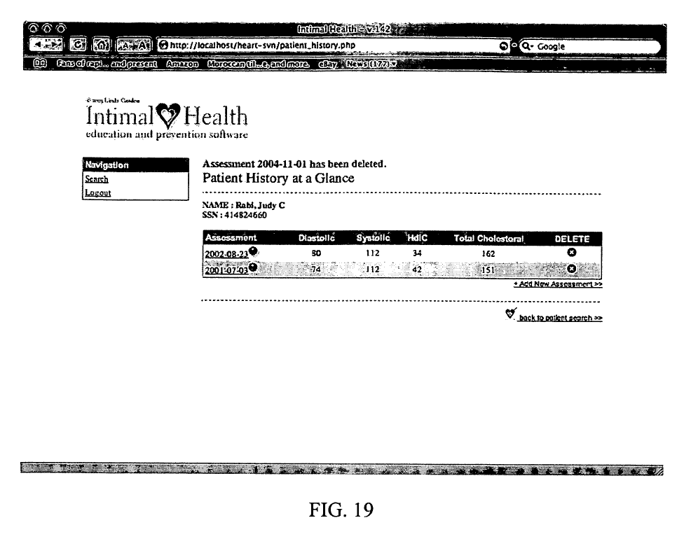

The physician may also have the opportunity to delete an assessment from the patient record at this time. As shown in FIG. 18, the deletion process is similar for almost all elements the user may delete. A confirmation screen can be displayed explaining what will be deleted and giving the user a chance to cancel the delete operation. As shown in FIG. 19, if the user decides to confirm the deletion process, they can be returned to the previous screen and an acknowledgement message can be displayed. In some instances, the deletion of medical records can be problematic. Accordingly, a log of all deletion requests can also be maintained. In one embodiment, a separate log of all database requests may be maintained by the system if desired. In another embodiment, changes made to individual records in the medical record database can be “stamped” with current time and the user who performed the action.

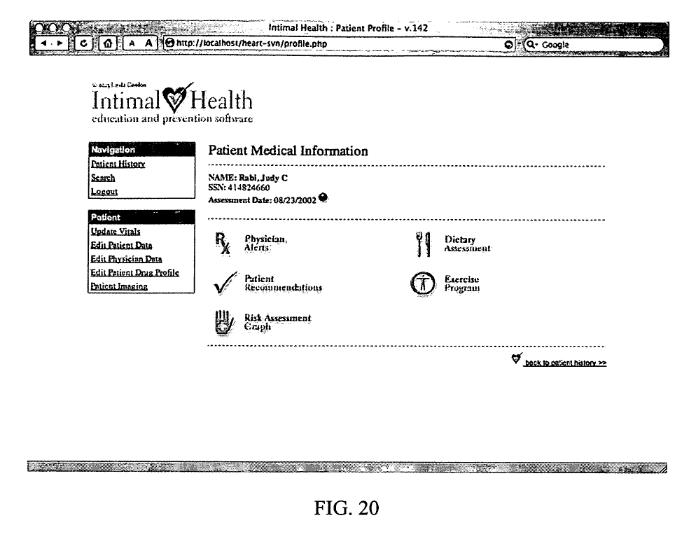

Once a physician selects a specific assessment to work with, unique identifier for that record can be placed in the user's persistent session file. This unique identifier is not changeable by the user nor is it modified by the system after the initial creation of the record. Even if the date of assessment is changed, such as, by subsequent office visits, this identifier remains consistent. As shown in FIG. 20, once the assessment identifier is stored in the user session, a patient profile screen can be displayed from which the physician can perform a number of actions. From this screen, a physician may update patient vitals, edit patient data, edit physician data, edit a patient drug profile, or view patient imaging. Patient imaging can include archived video, still images and/or radiographs.

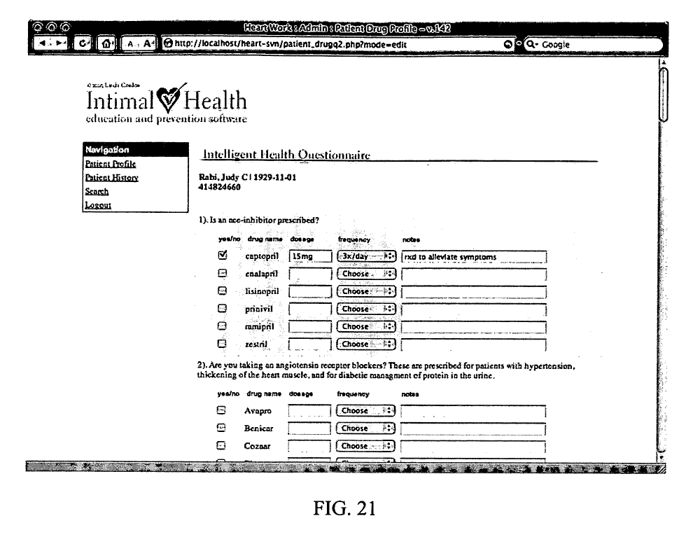

As shown in FIGS. 21-25e a patient's medical records can be updated, edited or created and a physician recommendation can be generated. FIG. 21 shows an example patient drug questionnaire. This questionnaire can be reached either through the assessment process or the patient profile, and presents the physician with a series of questions. Each question can relate to a specific classification of information such as, for example, medications such as statins, beta blockers, ace inhibitors, high blood pressure and the like. The questionnaire can generate a list of subsequent specific items depending on the previous series of questions. For example, if a family of medications is selected, a subsequent question may identify specific drugs within each class. The user would then select any of the medications and notes regarding the effects of the medication, dosages, and so forth may also be entered. These notes, along with additional information about the medication such as potential adverse effects can be displayed. Potential drug interactions and warnings can also be displayed on the assessment report page.

The physician may also access a screen identifying demographic information for a particular patient. This page, previously shown in FIGS. 15 and 16, can compile answers to all the questions that have been answered with existing information from the database. The physician may make changes to any element on this form. Additionally, this allows the physician to update patient address and other vital information on their own or from any downloadable web browser.

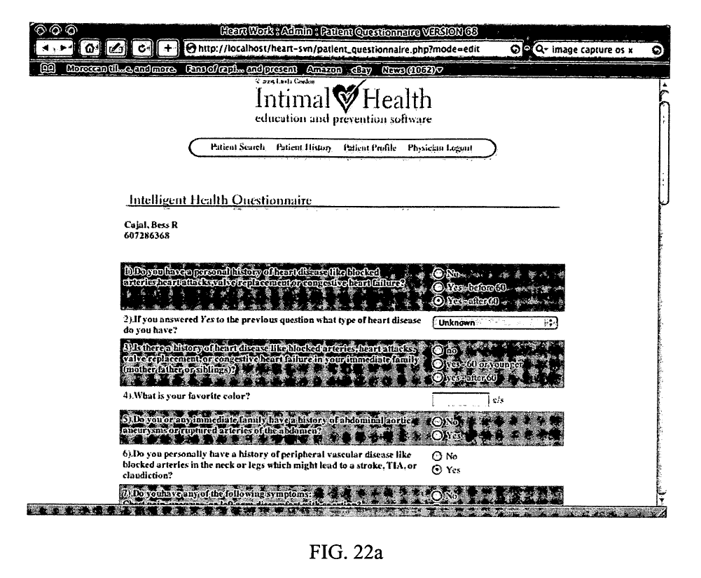

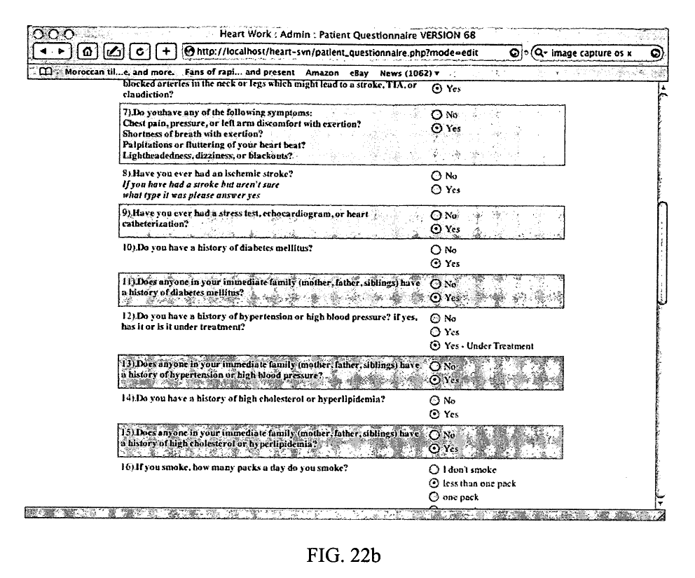

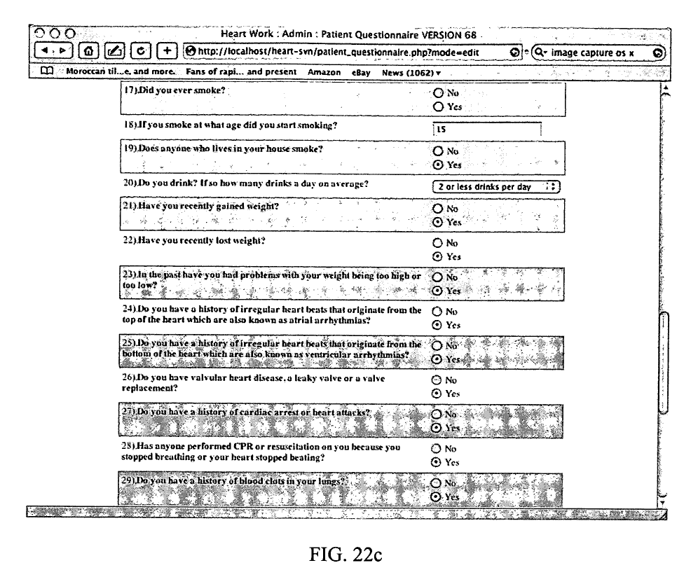

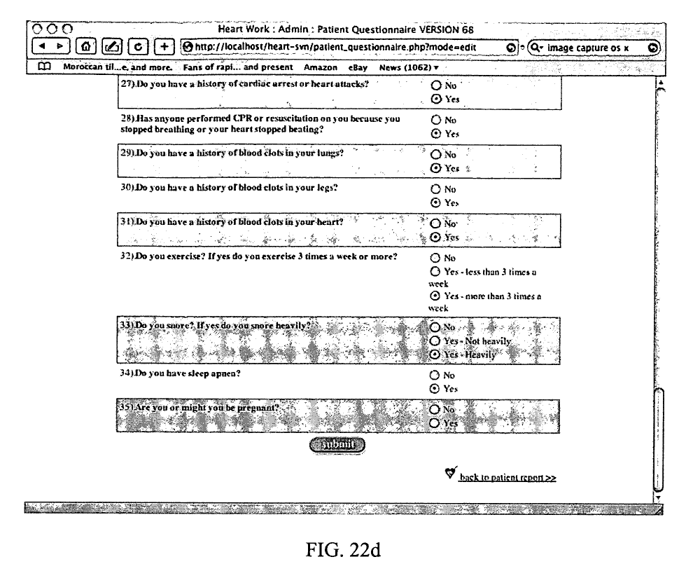

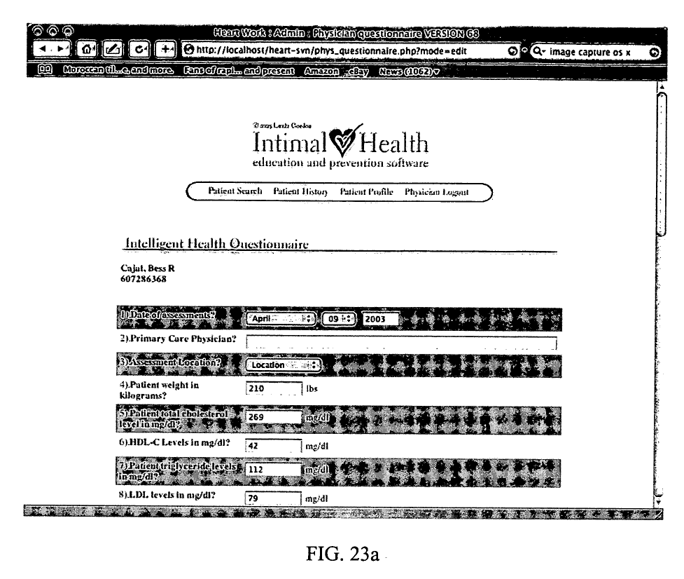

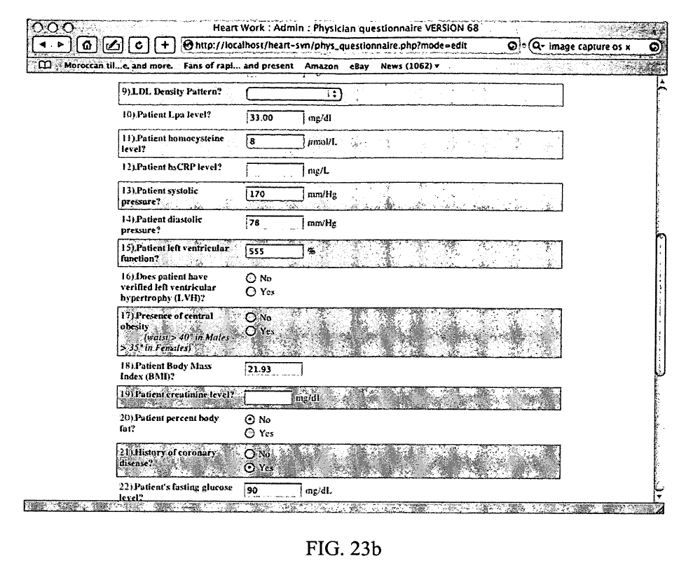

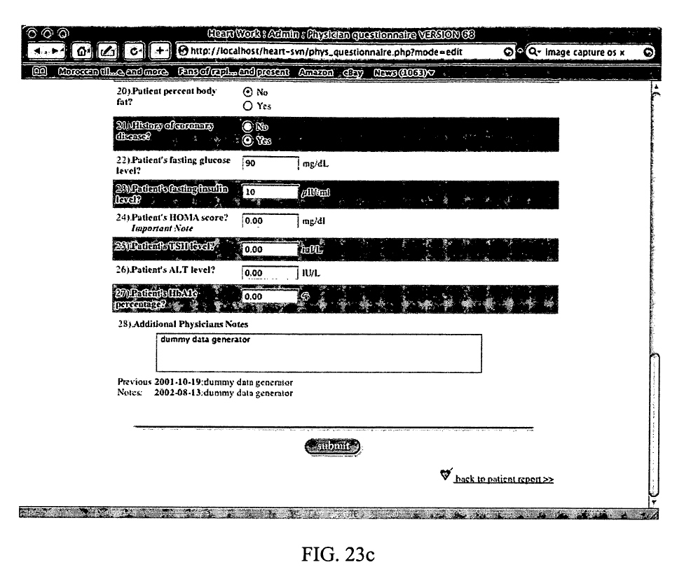

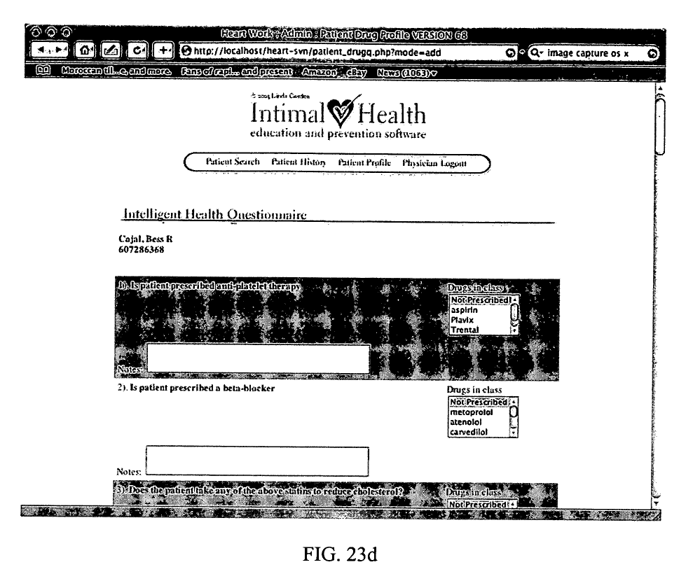

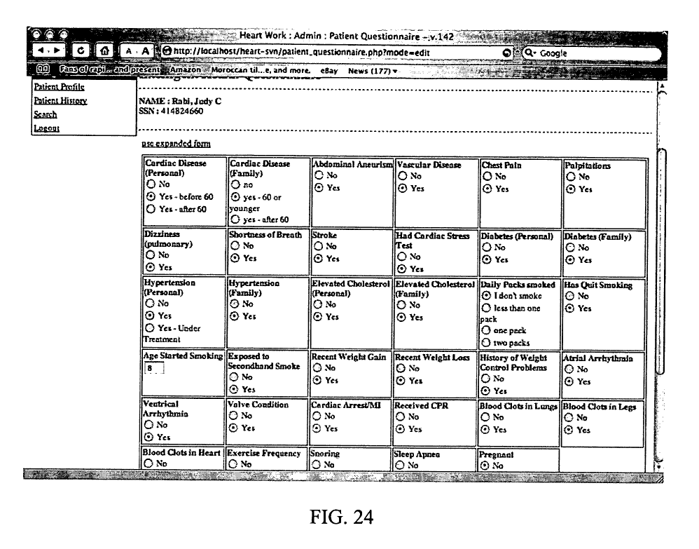

As shown in FIGS. 22a to 22d, a patient medical history questionnaire can be completed by either a patient or a physician working from the patient's existing medical history. The data related to the medical history may also be loaded directly into the database by an external process. Hospitals or other physicians' offices may communicate electronic records including photographs, radiographs, or charts indicating previous treatments. A physician questionnaire shown in FIGS. 23a to 23d can be dynamically generated from a database that contains all of the question elements. These can include the question itself, the class of question (medical history, lab values, etc.), the question type (text entry, radio button), formatting information, answer choices, and the database field where the answer to the question will be stored. It can also contain information on the layout of the data in the final assessment report and so forth. The questions database can be highly flexible and act as an interface between the system data and the processing algorithms. As will be shown later, questions may be easily added, modified, and deleted by the user. This gives the system a high level of flexibility to meet different requirements of different installation sites. While the results of these user created questions can then be displayed on the assessment page, they cannot be incorporated into assessment algorithms as of this time. Physician data can be acquired through the use of direct examination, blood test, physiological test, and so forth. In one embodiment, this page is only available to users logged in as physicians. The physician may reach this page either through the process of entering an initial or new assessment, or from the patient profile. The physician medical test questionnaire can allow a physician to update an assessment performed on a specific date with lab values that can be returned later. As is the case with the patient medical history questionnaire, the physician medical test questionnaire is generated dynamically from a questions database. Data can be entered into the physician medical test questionnaire through the form shown in FIG. 24 or loaded directly into the database through an external process. In either situation, a physician can edit data through this form. The questionnaire can also include a “freeform note” question. This can be a large text entry area in which a physician can add any sort of textual information related to the patient that they see fit. All notes from the previous assessment will also be displayed here. FIG. 23d shows an example of a drug interaction questionnaire. On this page, which can be reached either through the assessment process or the patient profile, the physician is presented with a series of questions. Each question relates to a specific class of medications—for example, statins, beta blockers, ace inhibitors and high blood pressure. The question then lists the specific drugs in each class. The user would select any of the medications the user has been prescribed. Notes regarding the effects of the medication, dosages, and so forth may also be entered. These notes, along with additional information about the medication, such as adverse effects, may be displayed on an assessment report. In one embodiment, the system includes an additional method, such as a message to the user, to warn the user of possible drug interactions.

Questions included in a questionnaire can include more than simple true false answers. For example, a question asking “Do you have a history of hypertension or high blood pressure?” can include the answers “No”, “Yes”, and “Yes, Under Treatment”. Likewise, a question asking “If you smoke, how many packs a day do you smoke?” can include the answers “I Don't Smoke”, “Less Than One Pack”, “One Pack”, and “More Than One Pack”. Questions contained on the patient medical history questionnaire can also include text entry boxes and drop-down menus. This screen can be generated dynamically from question elements and does not need to be hard-coded. By responsively generating targeted questions, the system can be easily adapted to different medical practices.

It should also be noted that the order the questions appear in are also entirely under the control of the local system administrator. Thus, emphasizing one group of questions can be determined by question orientation and order. The patient medical history questionnaire can also include dependent questions. Thus, each question has the question of only being displayed if other previous gathered data is true. In this case, a dependent question asking if the patient might be pregnant will be displayed only if the patient has indicated that they are female. Other questions may be tied to race, medical history, lab values or other data.

As shown FIG. 24, a questionnaire may be presented in a compact table format with abbreviated questions. This format is often preferable to users familiar with the system who do not need to read every word of all questions.

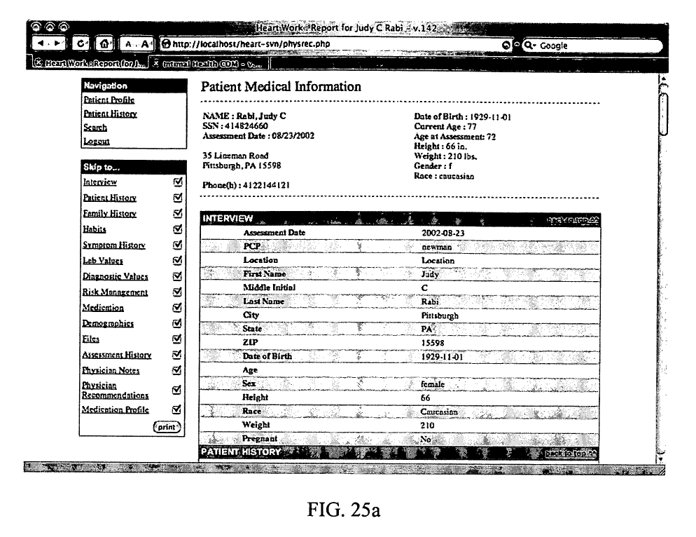

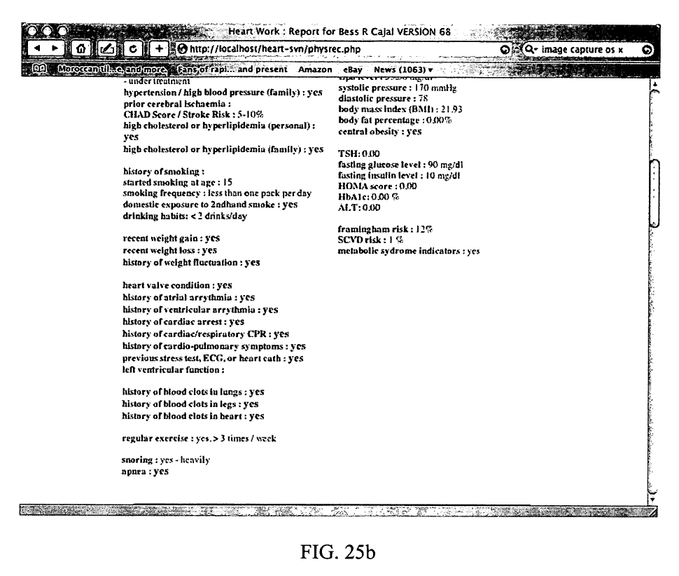

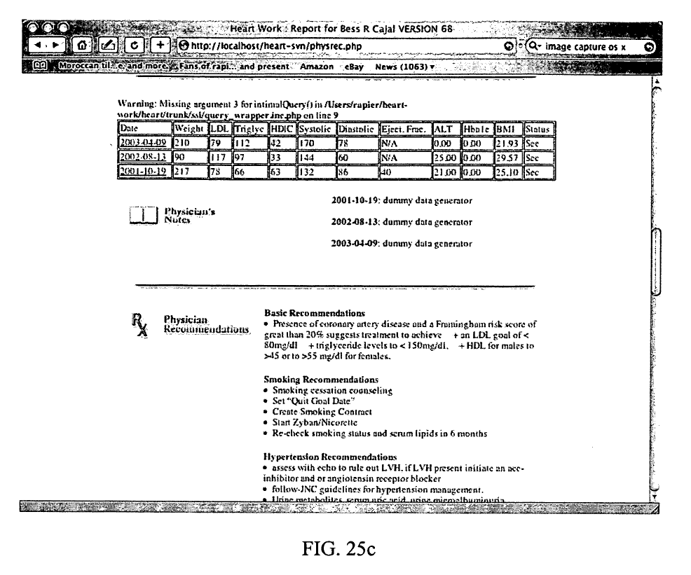

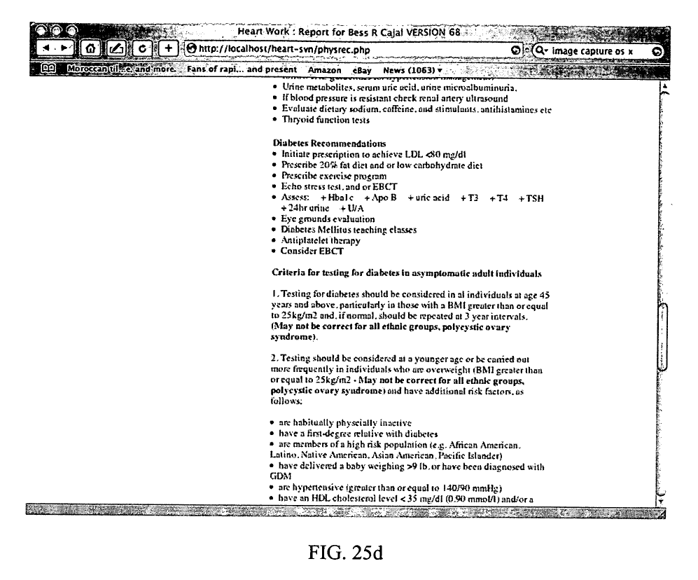

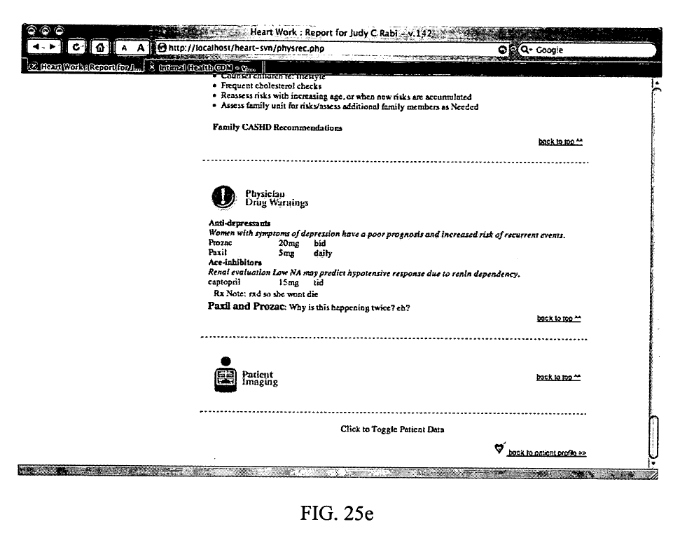

A physician can also enter data on a physician medical test questionnaire to produce a physician medical report shown in FIGS. 25a to 25e. As shown, a physician's alert screen may be included in the system. The physician alert page is analogous to the patient recommendation page described below, however, it provides additional information specifically geared toward physicians. This can include potential drug interactions, lab values, details from the patient medical history, and values computed by the system, such as the Framingham Risks Score, the HOMA value, body mass index, and the like. The physician alert page can also include results from the patient history, lab values, and computed values from the system.

In one embodiment, results of any questionnaire that are answered in the affirmative can be highlighted to aid in the rapid assimilation of data by the physician. In one embodiment, a history element and a note element can be included in the physician's alert page. The history element can include a grid-based display of critical patient information and lab values tracked over multiple assessments allowing the physician to quickly gage the efficacy of treatment. The notes element can compile all of the physician's notes and observations from previous assessments.

If a patient's lab values, history, or computed values indicate the presence of recognized conditions, the standard treatment protocol for that condition will be displayed in the physician recommendations. Even if the patient and/or physician isn't aware of the condition, the system will alert the physician that this particular patient meets certain criteria, thus prompting a recommended course of treatment and/or action. Also included in the physician's alert page is a physician drug warning section. This section can report on all the medication the patient is currently taking and/or previously taken. This report can include warnings to the physician, notes regarding the medication, and dosage information. Additionally, each drug can be compared to a predetermined set of interaction rules. If a prescribed medication is incompatible with the patient's current and/or previous medication regime, an interaction warning can be displayed. The physician also has the option of displaying or hiding any given section or subsection. This feature can be used to hide or displace selected portions of the screen to highlight or suppress information at the physician's discretion.

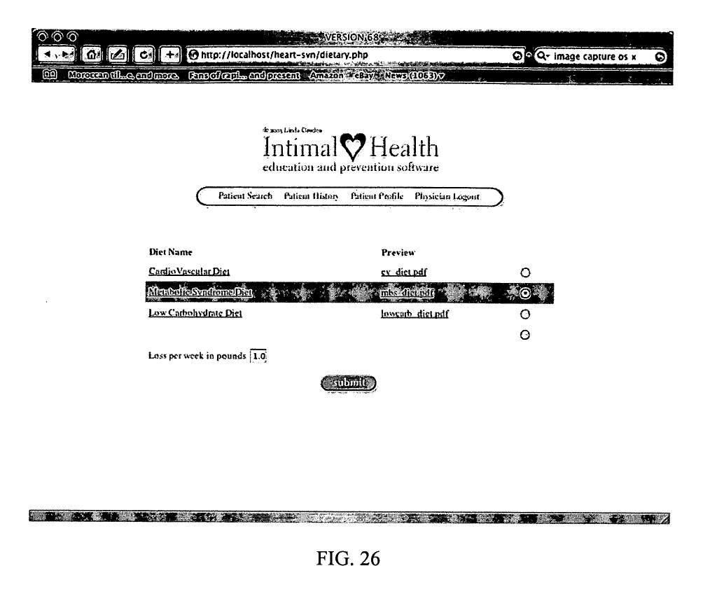

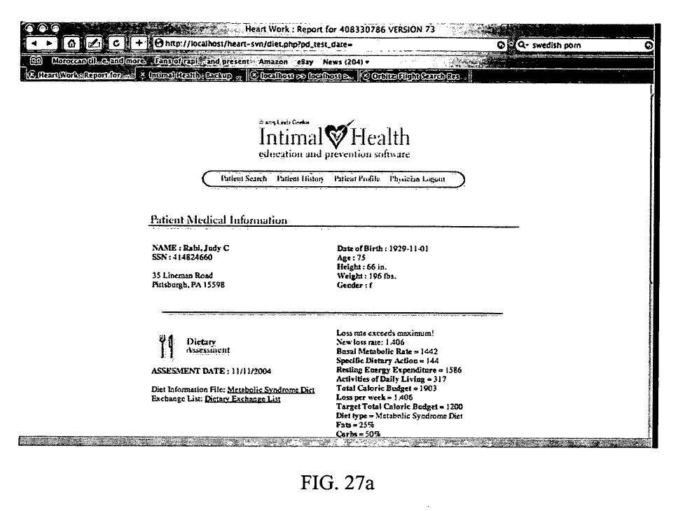

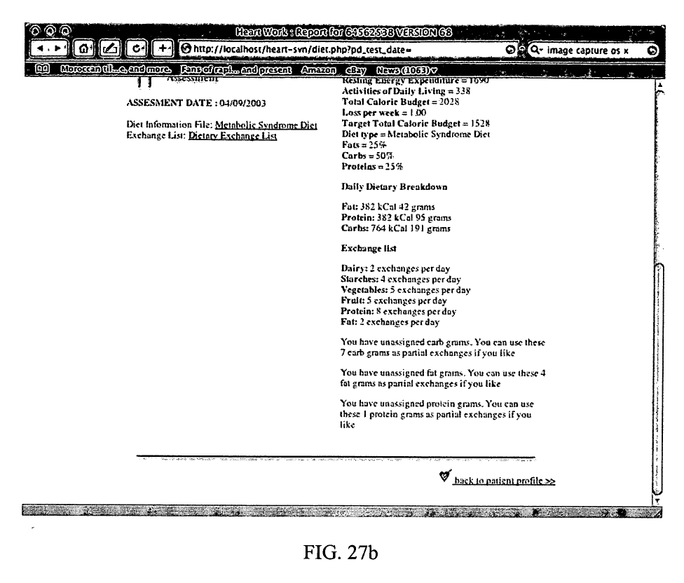

As shown in FIG. 26, a dietary input screen can also be displayed to the physician. A physician can assign the patient to any given diet. Commonly recognized diets can be included with the system, and a system administrator also has the opportunity to add additional diet options to the dietary input screen. The physician has the ability to enter the weekly weight loss goals on this page. In one embodiment, this data can be stored in the patient's profile for future reference. In another embodiment, if the physician has not yet selected a diet for a patient, a notice can be displayed suggesting that the patient contact the physician regarding their dietary needs. As shown in FIGS. 27a and 27b, a detailed dietary page can customize tailored results for the diet assigned by the physician based on the patient's age, weight, gender, activity level, diet type, and/or weight loss goals. The total daily caloric budget can also be computed by the system and calorie classifications can be broken down into fat, carbohydrates, and protein calories. Also included in the tailored dietary results page can be a number of standard dietary exchanges per food class. These can be based on accepted diabetic exchanges created by the American Diabetes Association and the American Dietary Association. In one embodiment, this page can include links to standardized dietary exchange lists and information files specific to the diet assigned by the physician.

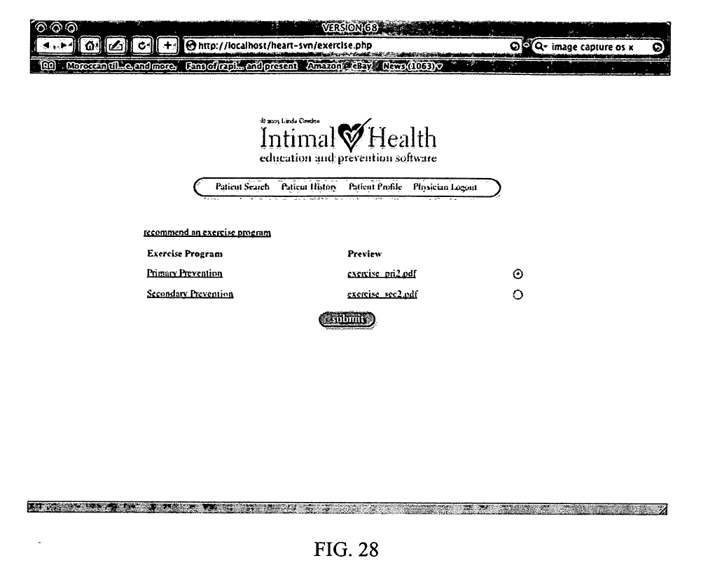

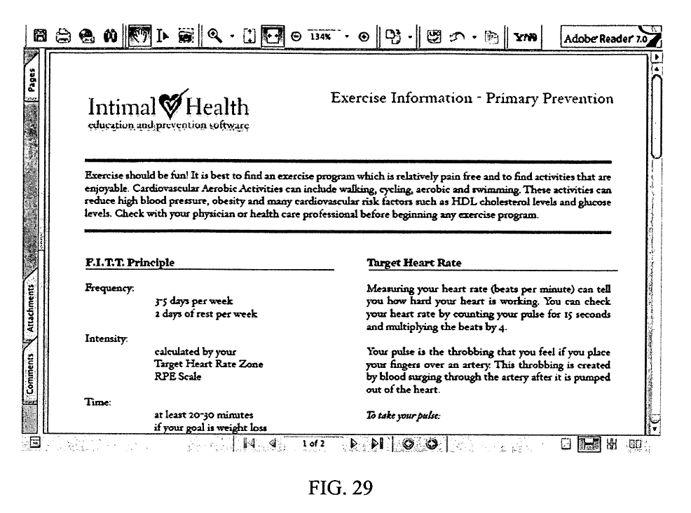

As shown in FIG. 28, an exercise page can be included within the system to allow a physician to assign an appropriate exercise program to a patient. The system administrator can create additional exercise programs specific to a given patient's needs. As shown in FIG. 29, a sample available exercise program is shown. Information in an available exercise program can be stored as a static PDF file. Additional PDF files may be uploaded to the system though an administrative interface.

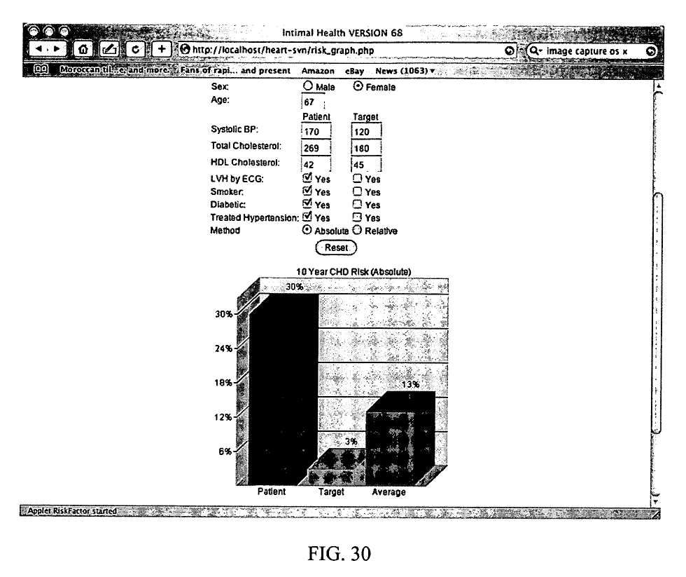

As shown on FIG. 30, the physician can access a Framingham Risk Assessment page. In one embodiment, the Framingham Risk Assessment page can make use of an embedded java application to visually display the current risk to the patient of a catastrophic medical event, such as a cardiac event. Risk assessment to the patient can be shown over a period of years. The specific risk factors for an individual are passed to the java application from the stored medical records of the patient. In one embodiment, the physician or patient may then examine the effect various risk factors have on the calculated risk.

### II Patient Access

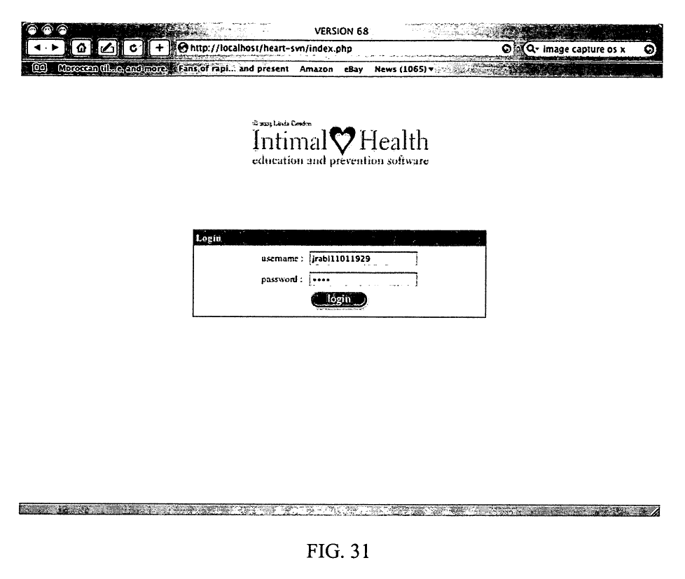

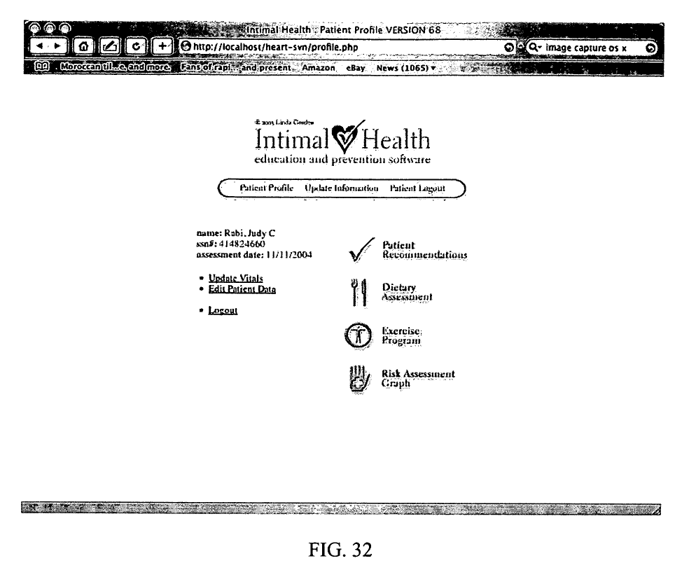

As shown in FIG. 31, a patient login screen is shown. In order to bypass the additional administrative overhead of creating user accounts and distributing user names and passwords, the system can use a standardized password for the patient login page. In one embodiment, the user name for patients can be the combination of the first initial, last name, and date of birth. As shown in FIG. 32, the patient access page allows patients to access information pertaining to their medical data and prescribed treatments as authorized by the physician. A patient questionnaire similar to the physician questionnaire is made available from this page. The patient questionnaire may be similar to the physician questionnaire except for a more limited ability to input data. The patient questionnaire may also be viewed in full or compact table format like the physician questionnaire. Pages such as the patient recommendations, dietary assessment, exercise program, and risk assessment graph may also be reached from this page. One of the primary goals of the patient profile is to educate the patient on appropriate treatment and lifestyle options. Reports generated for the patient can be completely determined by the administrator settings.

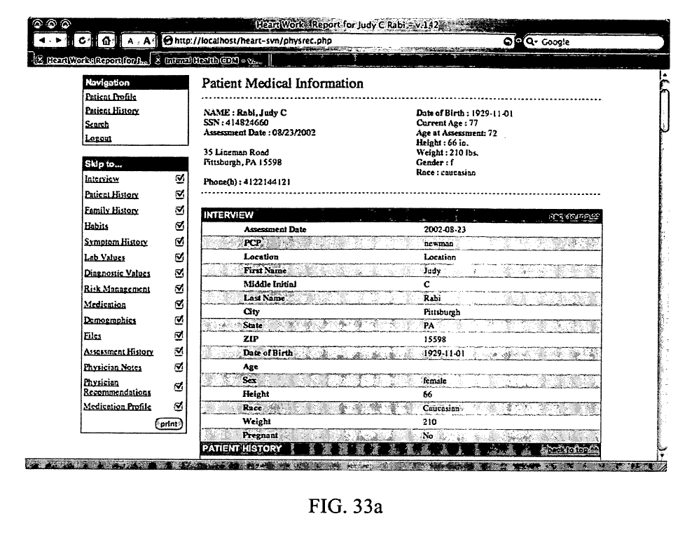

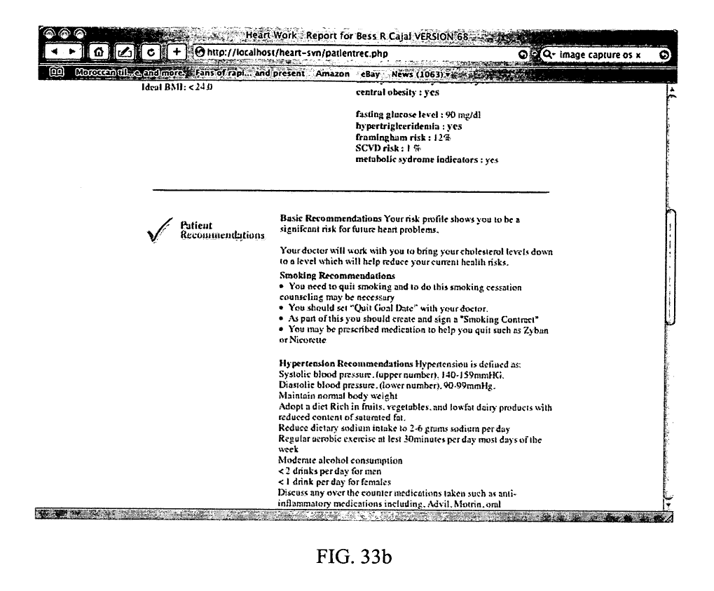

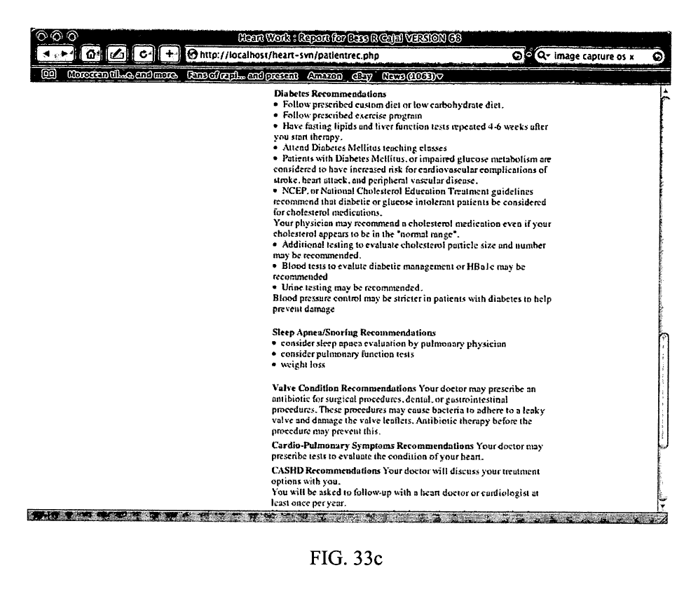

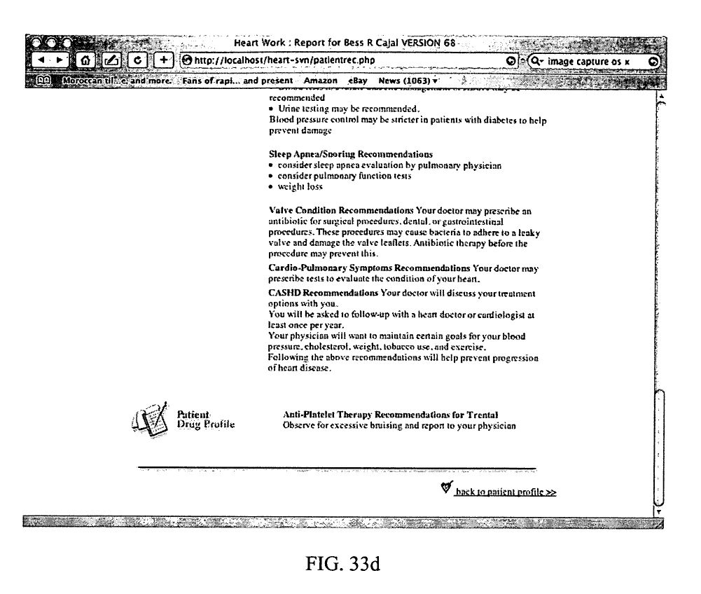

In one embodiment shown in FIGS. 33a to 33e, a patient can access a similar screen with less information than is available to a physician. Demographic data can be extracted from the database and used to populate the first section of information, providing such information as the name, address, date of birth, height, weight of the patient, and so forth. In one embodiment, a subset of lab values and test results can be displayed on the patient recommendation screen, such as in the second section. This section is not intended to release all available results to the patient, but provides a specific subset most relevant to the majority of patients. In one embodiment, patient values can be displayed in one column, and “ideal” values for these results can be presented in another column. The specific recommendations displayed to the patient can be dependent on the results of various tests and the patient's own medical history.

A specific routine that determines the recommendation set displayed to a patient can follow widely accepted protocols and standards for diagnosis and disease management. These recommendations can be written specifically as patient educational material and instructions. An administrator of the system can easily modify the content of the recommendations, such as through web-based interface. This allows the system to remain current without having to rely on frequent updates and revisions from the system company. In another embodiment, the recommendation text can be formatted with standard HTML markup and can even include JavaScript, java, frames, and PHP code. While this may not be applicable for an average administrative user, it does allow an advanced user to extensively modify the behavior of the system to suit more specialized needs. A patient drug profile, shown in FIG. 33d, can be included in the patient recommendation screen in which information regarding adverse reactions, standard dosing information, and the like, is displayed. In one embodiment, only currently prescribed medication is displayed. Dosing instructions may also be included in the section.

### III Administrator Access

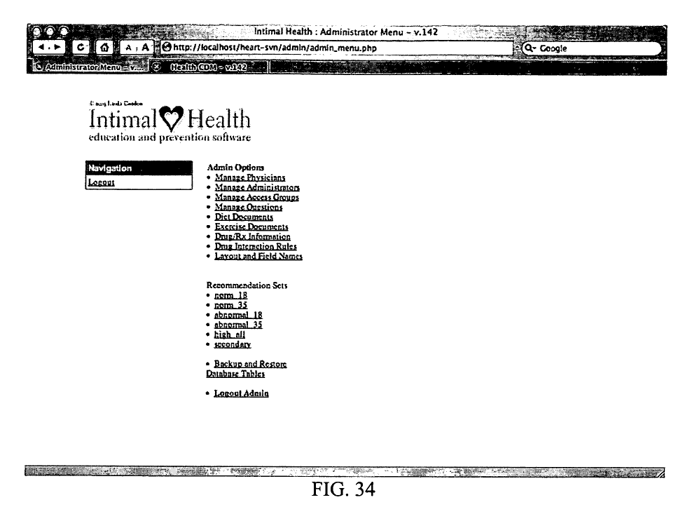

As shown in FIG. 34, administrators may also access the system to add or remove patient and/or physician users, to edit, add, and/or delete diet and exercise programs, pharmaceutical information, questions for the patient and physician questionnaires, drug interactions, and recommendations provided to both the patient and physician. Administrators may also backup and restore database tables. As shown in FIG. 34, each individual data input is characterized by the appropriate page layout. Page layouts can include patient history, family history, symptom history, habits, interviews, and the like. For example, fields pertaining to personal cardiac disease have a page layout on the patient history.

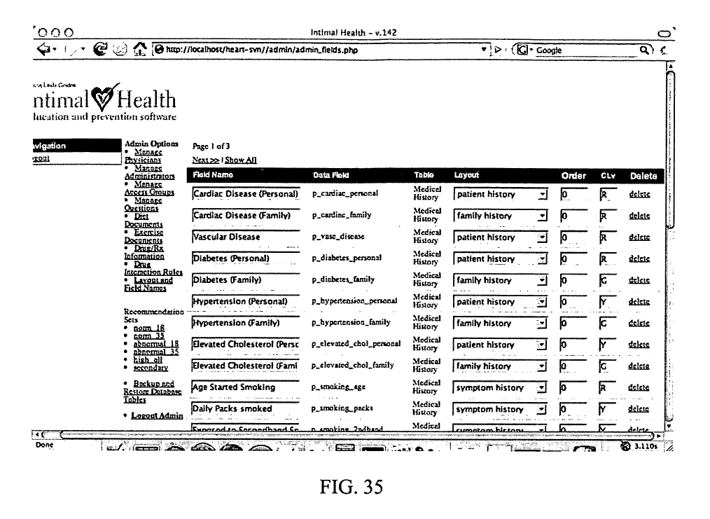

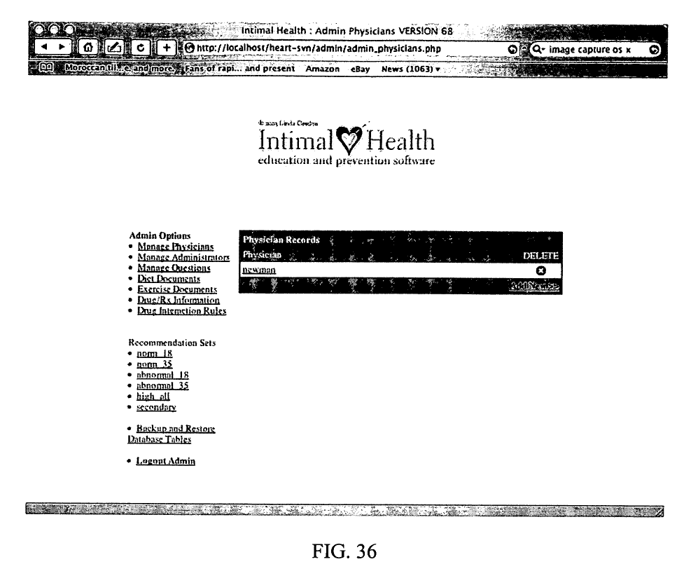

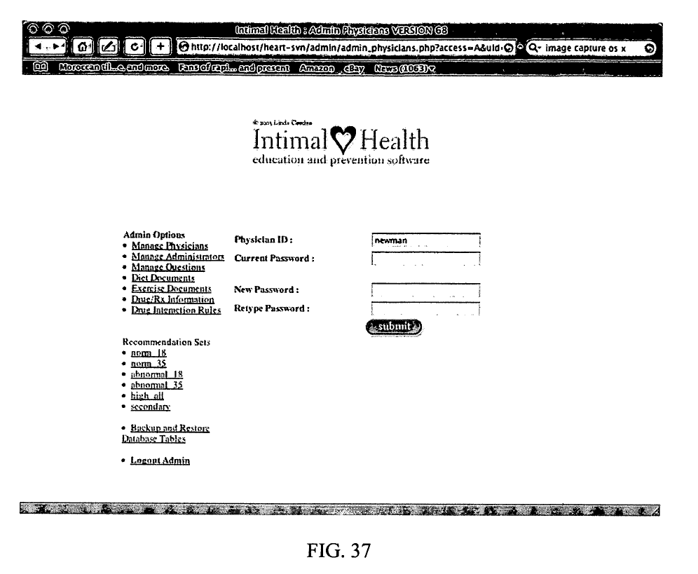

As shown in FIG. 35, an administrator can change the appearance of a page by altering the organization of a page layout including column separation, column layout, and menu options. As seen in FIG. 35, an administrator may also change the criticality of data within a given field by change the input in the CLv column. An R, Y or G in this column will indicates the color-code that will show up on a report should data from this field be incomplete. As shown in FIG. 36, an administrator may add and delete users in the physician class. As shown in FIG. 37, the administrator can change and/or assign passwords to the physician. In one embodiment, passwords can be stored in the access control list as a one-way encrypted hash value. Similarly, an administrator can add new physicians by assigning a user name and an initial password. An administrator can also add and delete users of the administrator class. The method of changing passwords and adding new administrators can be identical to the physician management process.

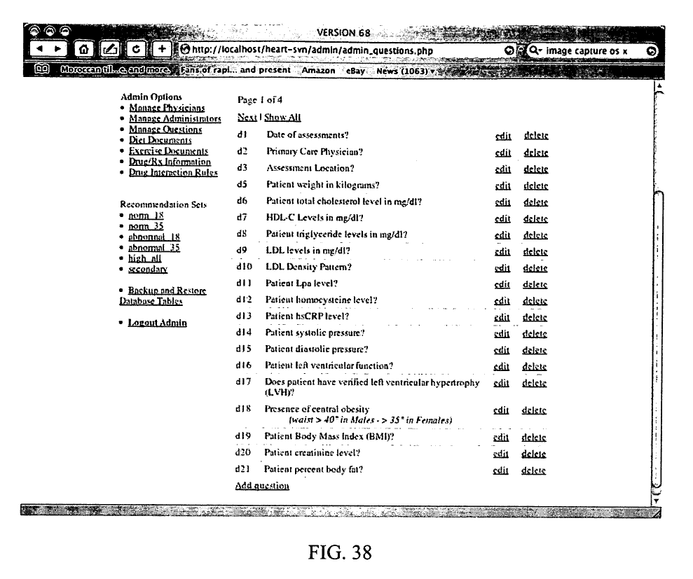

As shown in FIG. 38, a questions management page can be included in the system. A questions management page can provide a list of all of the questions used in the patient and physician questionnaires. In one embodiment, all patient and physician questionnaires are developed from the questions management page, while data fields can be added to the main patient database. For example, if a physician wanted to start collecting data on resting heart rate, then the administrator could add a question and associate it with a new database field. From here, the physician can search on this data, have it displayed on the patient or physician assessment reports, or almost any action that can be performed on the default database fields.

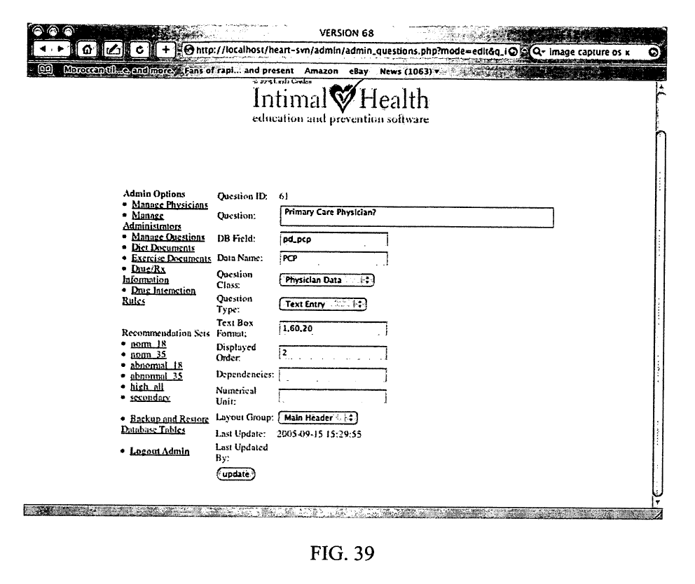

As shown in FIG. 39, the administrator can modify existing questions. An administrator can modify the test of a question, change the database field the question is associated with, give the data a “display” name to use on reports, assign it to a class (physician, patient, or image), or have it displayed in one of multiple formats (text, radio buttons, options list, and the like). Additionally, an administrator can change the order the questions will be displayed in. The administrator can also assign each individual question to a specific layout section on the patient or physician assessment report. The dependency fields can be used to determine if a question should be asked or not. For example, based on the dependencies, the question might only be displayed if the patient is female or of a specific race. Additionally, the last time the question was modified and who modified the question can also be recorded.

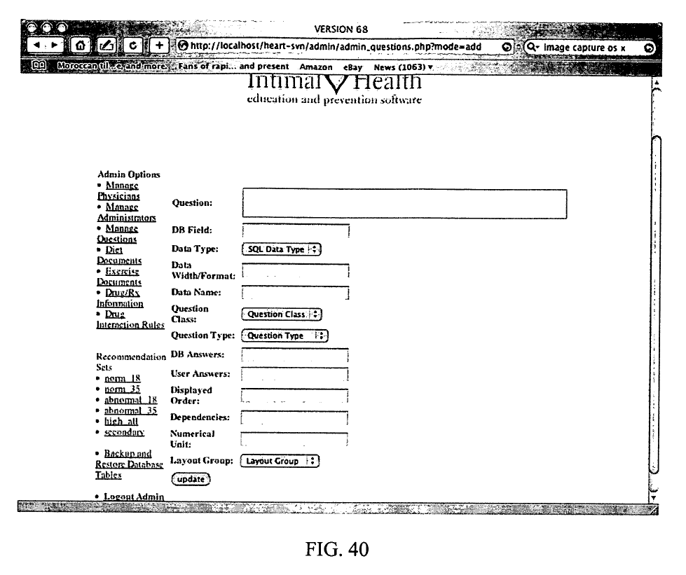

As shown in FIG. 40, an administrator can add new questions to the system. The primary difference between this page and the “edit question” page is that the administrator has to define a data field to store the user answers to the question. In one embodiment, the administrator can pick a name for the database field, select an appropriate SQL data type, and define the width of the data field. In other respects, this page can be substantially the same as the editing page.

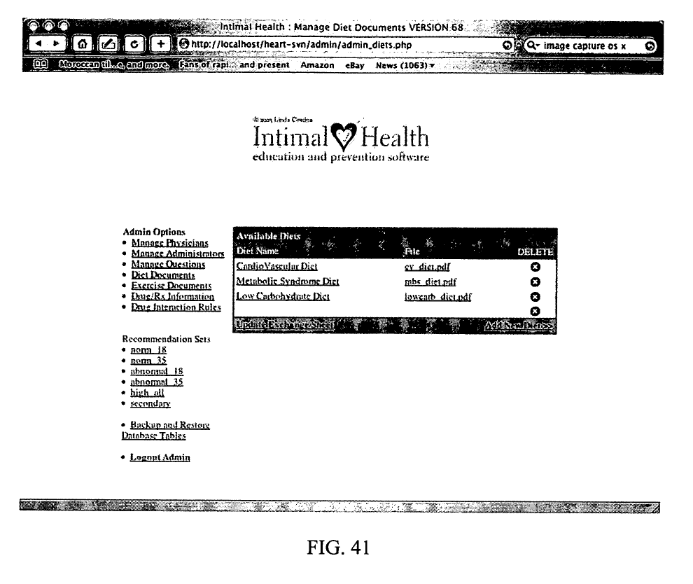

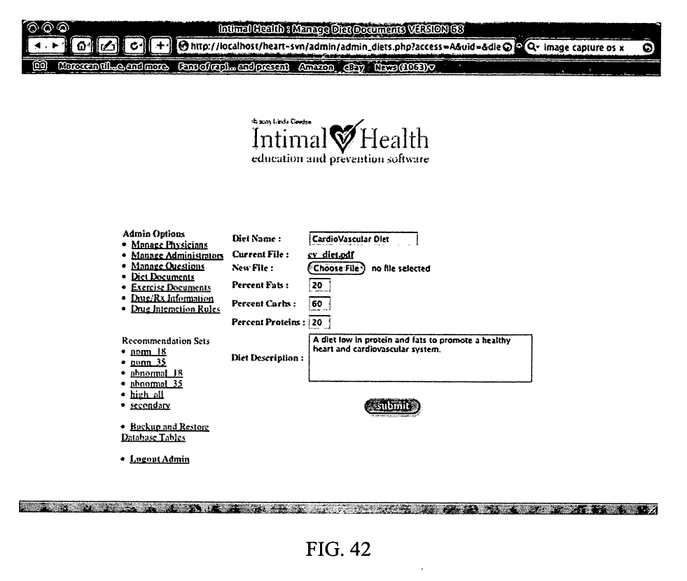

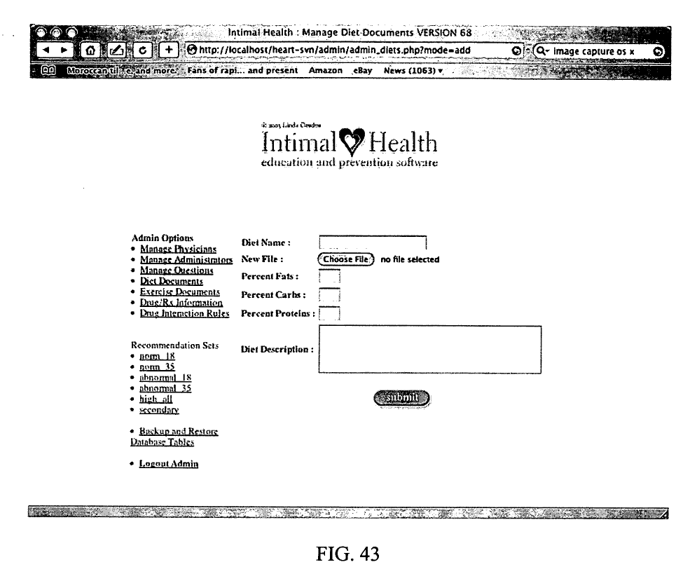

As shown in FIG. 41, a diet document administration screen can be included in the system. From this screen, an administrator can review or delete diets. An administrator can also edit existing diets, add new diets, and update the dietary exchange information sheets. As shown in FIGS. 42-43, the diet documents management page can include an editing section. The administrator can specify a name, the PDF file associated with the diet, the percentages of fats, carbohydrates, and proteins that make up the diet, and add a description of the diet.

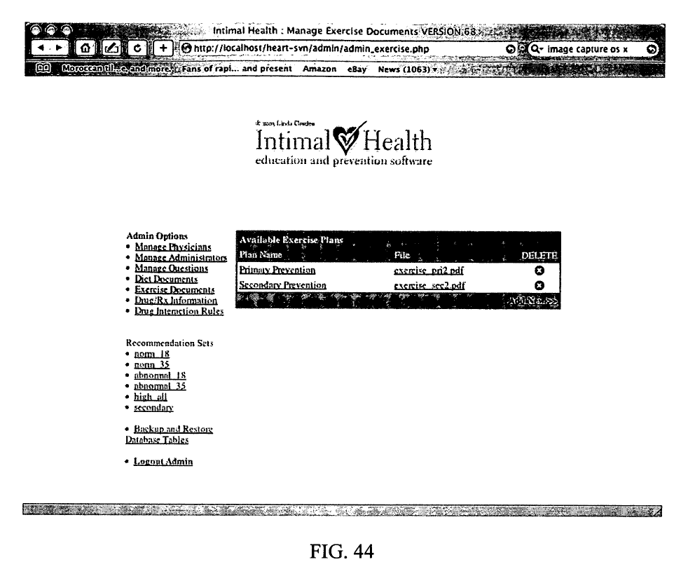

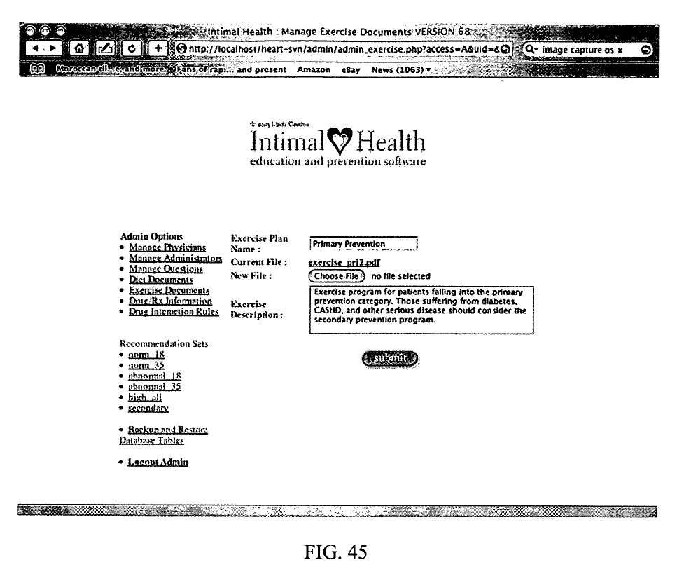

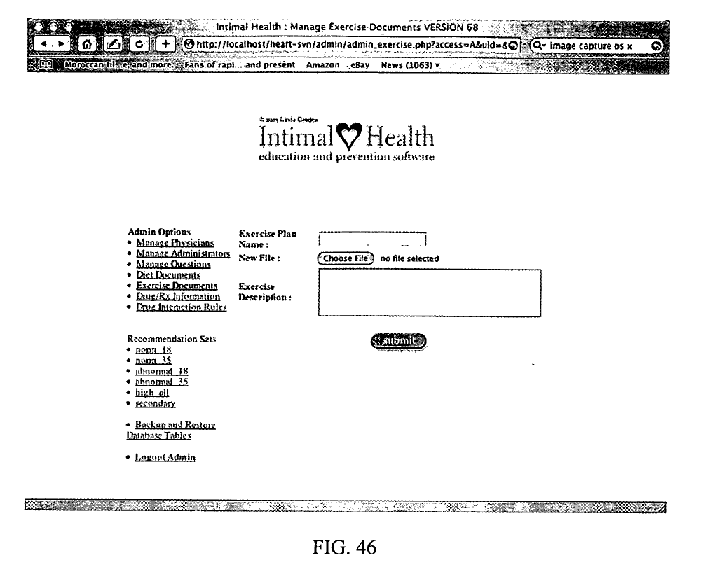

As shown in FIG. 44, an administrator may review or delete exercise documents. An administrator may also have the option of adding a new exercise document or editing an existing exercise document. As shown in FIG. 45, an administrator editing form for exercise documents can also be included in the system. An administrator is given the opportunity to name and describe an exercise program. Additionally, an administrator may upload a PDF document containing specific exercise programs. As shown in FIG. 46, an administrator may add a new exercise document in the system. This can function in a substantially similar fashion through the editing page.

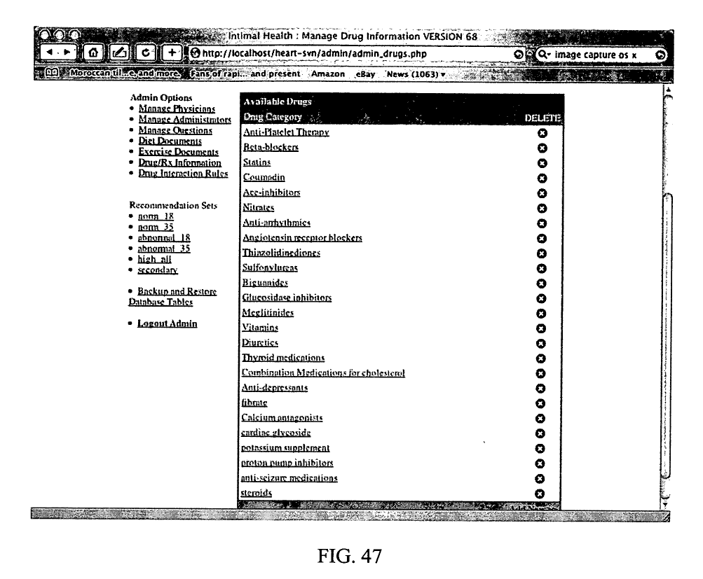

As shown in FIG. 47, an administrator can manage specific pharmaceutical drug classes. Drug classes can comprise any logical groupings of medications. In one embodiment, each class can hold information on one or more specific medications. For example, the “statins” group holds information on several medications that work in a similar manner to lower serum cholesterol. From this page, the administrator can add new drug classes, delete existing drug classes (and all of the medications that a class may contain), or chose to edit an already existing drug class. In another example, this information is used to dynamically generate the drug information form used to collect medication profiles from the patient.

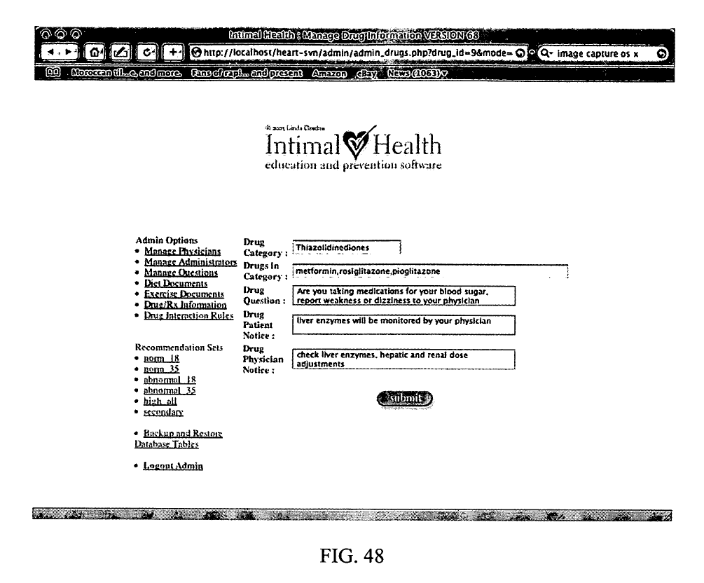

As shown in FIG. 48, an administrator can edit an existing drug class. An administrator can select the name of a specific drug class and then list all of the medications composing that class. An administrator can also enter the text of the question to be displayed on the drug information form, the notice that would be given to patients who are taking medications in this class, and finally the physician notice.

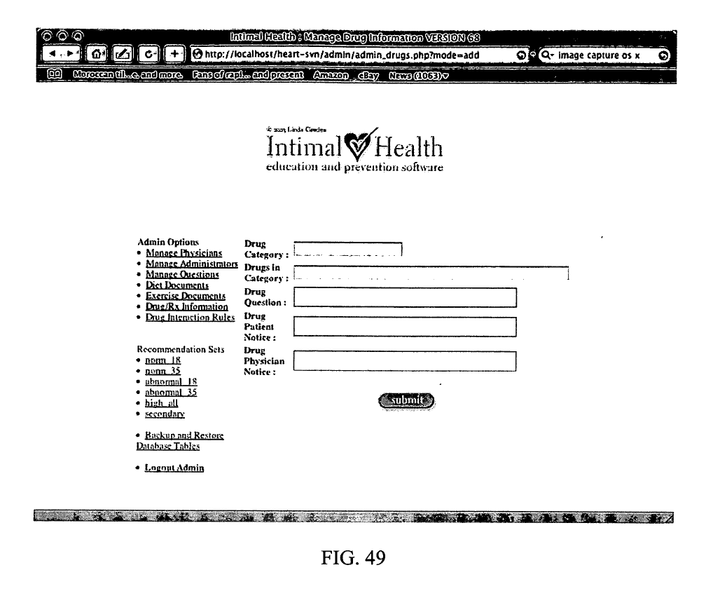

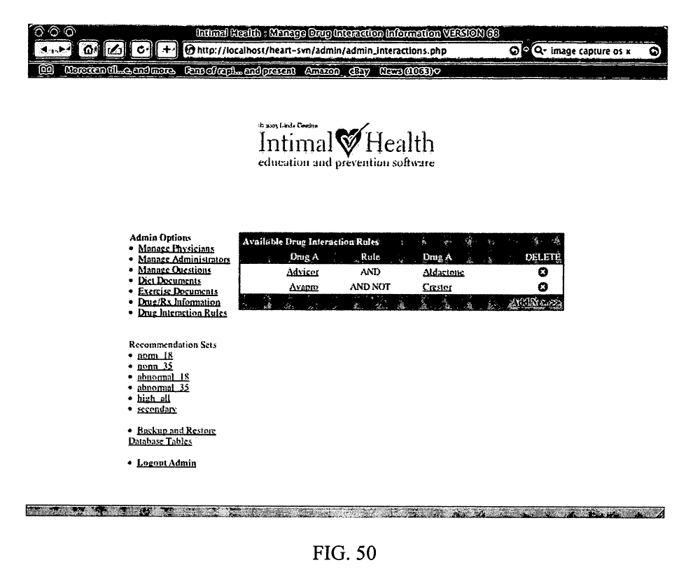

As shown in FIG. 49, the administrator can create a new drug class. Information contained in the new drug class can be inserted into the database as new information instead of an update on existing information. As shown in FIG. 50, the administrator may access and edit the drug interaction rule screen. In one embodiment, a database of rules can be created by the administrator using Boolean logic. The rules can be designed to warn the physician and/or patient if two or more incompatible medications are being, or not being, taken at the same time. For example, if two medications should be prescribed in tandem, and only one is listed in the patient's medication profile, the patient and/or physician will be warned about this. Likewise, if a patient's medication profile indicates that they are prescribed to antagonistic medications, a warning to this effect can be generated.

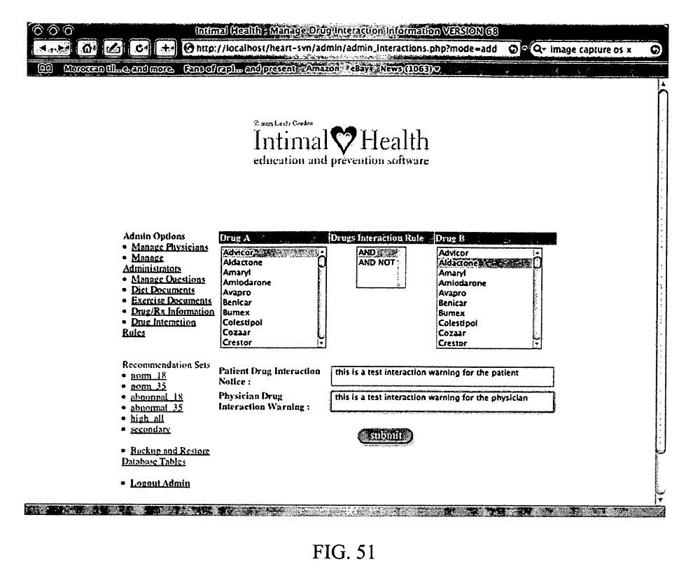

As shown in FIG. 51, an administrator can edit or create a drug interaction rule. The administrative user can select a medication from the first column, an interaction (AND or AND NOT) from a second column, and a medication from a third column. The administrator can then enter the text of the notice to be presented to the patient and physician. In one embodiment, the routine stores the components in a database table from which the rule set is reconstructed as needed.

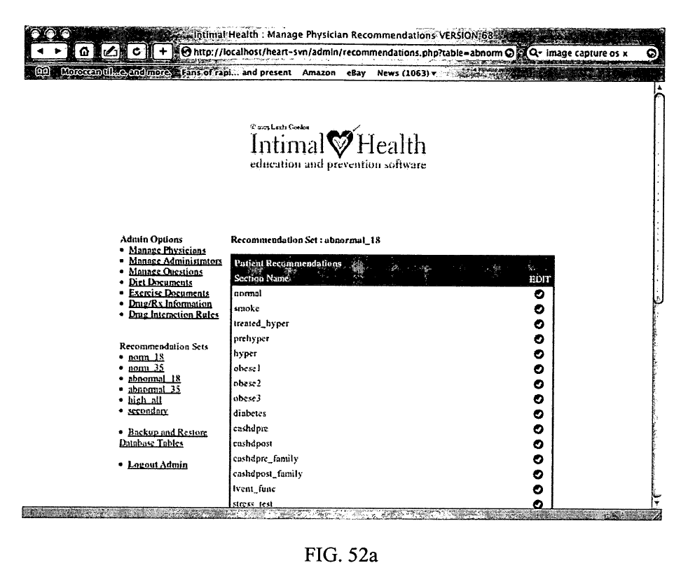

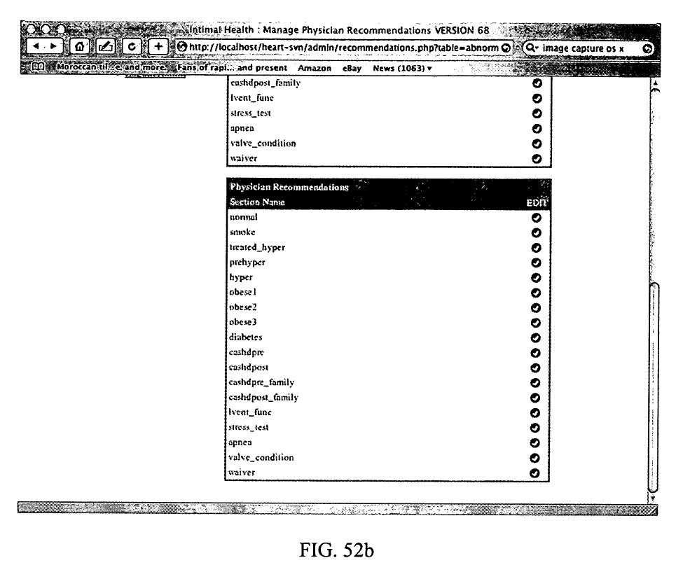

As shown in FIGS. 52a and 52b, a recommendation set management page can be included in the system. Users are broken down into classes of patients, depending on various medical characteristics. For example, a 45-year-old patient with a normal lipid profile and no history of heart disease or diabetes, could be placed in the “norm 35” class. Each class contains a number of recommendations that correspond to specific disease profiles. Each patient can be evaluated for inclusion or exclusion from a specific disease profile based on their characteristics. As treatment protocols change or if an individual practice or medical system has a unique protocol for that specific disease, the administrative user can change the recommendations to meet changing needs. Patient and physician categories may each contain different treatment information and instructions. By choosing one of these categories, the administrator can edit the text presented to the specific users.

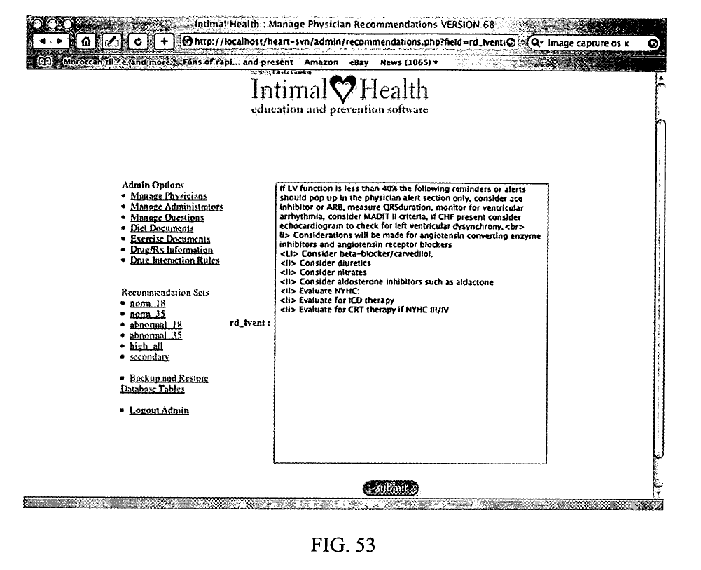

As shown in FIG. 53, an administrative user can modify the text of a recommendation. In one embodiment, standard HTML markup may be used to format the information. Additionally, JavaScript, embedded Java applications, PHP, and other server side instructions can be added using this interface.

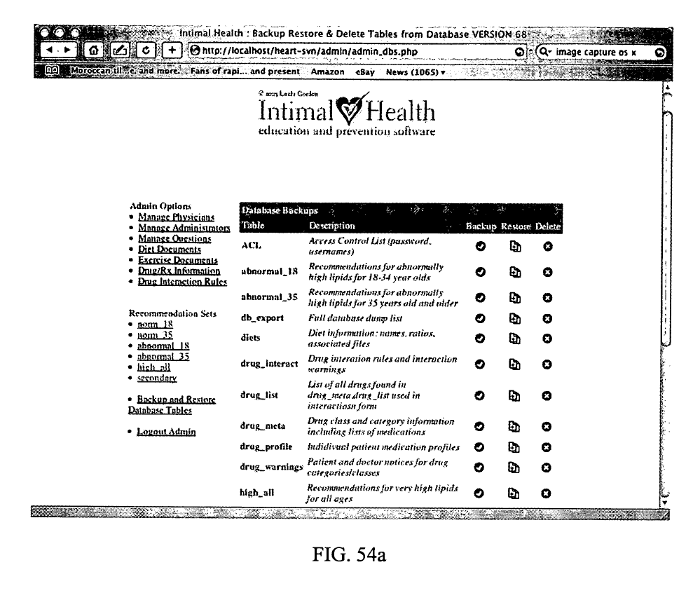

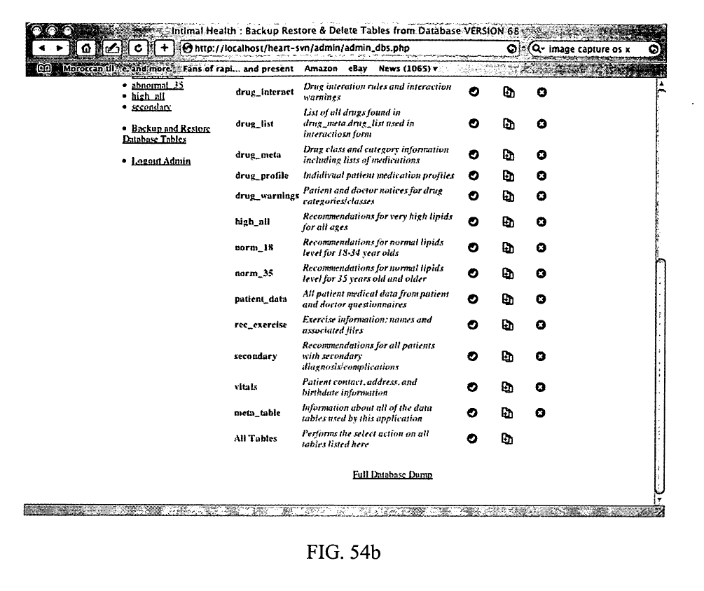

As shown in FIGS. 54a and 54b, maintaining data integrity is vital to the application. Due to the fact that there are numerous database tables and users, especially administrative users, the user may under the right circumstances render portions of the software inoperable. Therefore, a data backup and restore functionality can be built into the software. In one embodiment, this is in addition to any other backup procedures the user might elect to use. Patient identifying data can be stored in an encrypted format that may only be unlocked by the user defined encryption key. In the event one of these files are misplaced, stolen, or corrupted, their utility is strictly limited. From this page, the administrative user can backup individual tables, restore individual tables, or delete the contents from individual tables. The administrator may also backup all tables or download the entire database. A “full database dump” option is included in the system.

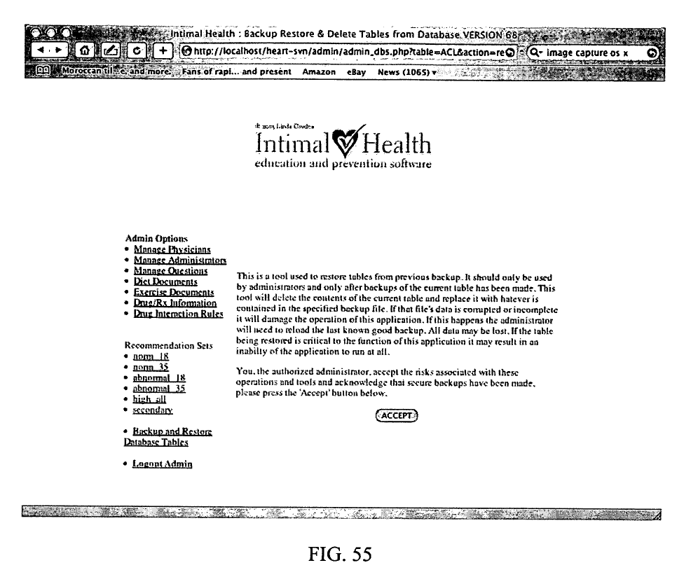

As shown in FIG. 55, if an administrator elects to restore a particular data set, they are presented with a warning informing them that the restore function will overwrite any existing data in the table. The administrator may accept this addition before picking the specific backup to restore. If the administrator opts to delete all the information from a table, they are also warned that all information in that table will be lost, and that a backup should be created first.

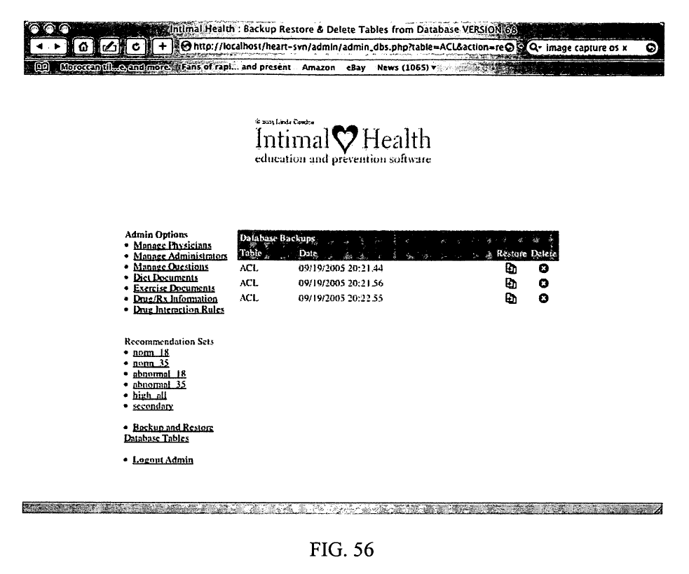

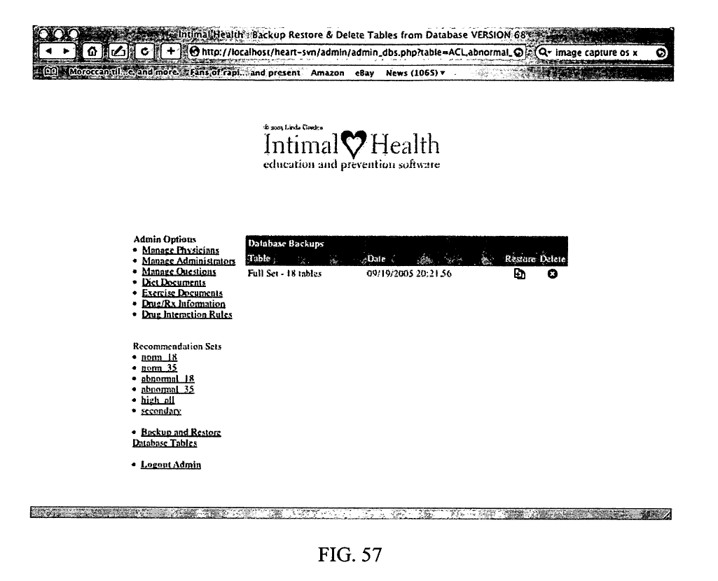

As shown in FIG. 56, if the administrator accepts the function, they are presented with a date-sorted list of data tables that can be restored to the operating database. An option for deleting such tables can also be included. As shown in FIG. 57, if an administrator has backed up all of the data using the all tables option, the administrator will be presented with “full sets” of tables to restore. This will restore all of the backed-up tables from a specific “all tables” backup operation.

Although the above-example of the present system has been described with reference to heart conditions, it is contemplated herein that various other medical diseases can be managed and treated with the system of the present invention. Other medical practices that could utilize the electronic disease management system of the present invention include general internists, obstetrician/gynecologists, pulmonologists, urologists, neurologists, ophthalmologists, ear-nose-and-throat specialists, orthopedists, urologists, podiatrists, psychiatrists, gastroenterologists, pediatricians and the like. It is also noted herein that although the above-example of the present system has been described with reference to drugs and medical devices that are specific to heart conditions, any suitable drugs, medications, pharmaceuticals, and/or medical devices could be potential input fields and/or potential physician recommendations in accordance with the present electronic disease management system.

Whereas particular embodiments of this invention have been described above for purposes of illustration, it will be evident to those skilled in the art that numerous variations of the details of the present invention may be made without departing from the invention.

What is claimed is:

1. A computer-assisted disease management system, comprising:
a computer system capable of receiving a plurality of patient information inputs and a plurality of medical standards inputs; and
means for comparing the patient information inputs against the medical standards inputs to produce a diagnosis output.

2. The computer-assisted disease management system of claim 1, wherein the patient information comprises previous patient information.

3. The computer-assisted disease management system of claim 2, wherein the previous patient information comprises records from multiple previous treating physicians, previous hospital records, previous prescribed drugs and/or previous prescribed medical devices.

4. The computer-assisted disease management system of claim 1, wherein the patient information comprises current patient information.

5. The computer-assisted disease management system of claim 4, wherein the current patient information comprises real time information provided by a patient, notes and diagnosis render by a current treating physician, currently prescribed drugs and/or currently prescribed medical devices.

6. The computer-assisted disease management system of claim 1, wherein the patient information comprises previous patient information and current patient information.

7. The computer-assisted disease management system of claim 6, wherein the previous patient information comprises records from multiple previous treating physicians, previous hospital records, previous prescribed drugs, and/or previous prescribed medical devices and wherein the current patient information comprises real time information provided by a patient, notes and diagnosis render by a current treating physician, currently prescribed drugs and/or currently prescribed medical devices.

8. The computer-based disease management system of claim 1, wherein the medical standards inputs comprise accepted treatment regimes and/or medical literature.

9. The computer-assisted disease management system of claim 1, wherein the computer system comprises a central server in data-communication with at least one remote system.

10. A computer-assisted disease management system, comprising:
a computer system capable of receiving a plurality of patient information inputs and a plurality of medical standards inputs;
means for generating an interactive questionnaire in response to the previously entered patient information inputs; and
means for comparing the patient information inputs against the medical standards inputs to produce a diagnosis output.

11. A computer-implemented method of generating a recommendation output, comprising:
entering a plurality of patient information inputs into a computer system;
entering a plurality of medical standards inputs;
generating an interactive questionnaire in response to previously entered patient information inputs;
comparing the patient information inputs against the medical standards inputs; and
generating a recommendation output in response to the comparison of the patient information inputs and the medical standards inputs.

12. A computer-readable medium having stored thereon instructions which, when executed by a processor, cause the processor to perform the steps of:
comparing patient information inputs against medical standards inputs; and
generating a recommendation output in response to the comparison of the patient information inputs and the medical standards inputs.

13. A computer-readable medium having stored thereon instructions which, when executed by a processor, cause the processor to perform the steps of:
generating an interactive questionnaire in response to previously entered patient information inputs;
comparing the patient information inputs against medical standards inputs; and
generating a recommendation output in response to the comparison of the patient information inputs and the medical standards inputs.

14. An apparatus, comprising:
means for receiving a plurality of patient information inputs;
means for receiving a plurality of medical standards inputs;
means for generating an interactive questionnaire in response to the previously entered patient information inputs; and
means for comparing the patient information inputs against the medical standards inputs to produce a diagnosis output.

15. A system, comprising:
a processor; and
a memory in communication with the processor, the memory having stored thereon a set of ordered data and instructions which, when executed by the processor, cause the processor to perform the steps of:
generating an interactive questionnaire in response to previously entered patient information inputs;
comparing the patient information inputs against medical standards inputs; and
generating a diagnosis output in response to the comparison of the patient information inputs and the medical standards inputs.

16. A multi-user computer-based disease management system, comprising:
a data store for storing medical standards inputs and patient information inputs;
a computer system capable inputting and extracting medical standards inputs and patient information inputs from the data store;
means for allowing user initiated input of medical data into the data store;
means for allowing automatic input of medical data into the data store; and
means for allowing user interrogation and extraction of data from the data store.

17. The multi-user computer-based disease management system of claim 16, further comprising means for identifying a type of user of the system, wherein the type of user is a patient, a physician or an administrator.

18. The multi-user computer-based disease management system of claim 17, wherein the means for extraction of data from the data store comprises a dynamic multi-variable querying system when the user is a physician.

19. The multi-user computer-based disease management system of claim 17, wherein the computer system restricts or allows access to content in the data store based on the type of user of the system.

20. The multi-user computer-based disease management system of claim 16, further comprising means for generating an interactive questionnaire in response to patient information inputs.

21. The multi-user computer-based disease management system of claim 20, further comprising means for generating a diagnosis output in response to the comparison of the patient information inputs and the medical standards inputs.

22. The multi-user computer-based disease management system of claim 16, wherein the means for allowing user interrogation and extraction of data from the data store comprises a recommendation output.

23. The multi-user computer-based disease management system of claim 22, wherein the recommendation output is a physician recommendation or patient recommendation.

24. The multi-user computer-based disease management system of claim 16, wherein the means for allowing user initiated input of medical data into the data store comprises an HTML document which changes input options dynamically in response to user inputs.
